SEQUENCE LISTING (24 sequences)

SEQ ID NO: 1 - protein, 261 aa, Artificial Sequence
  Synthetic amino acid sequence Gel (recombinant gelonin)
ISEFGSSRVDLQGLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLFVDKDPK

SEQ ID NO: 2 - protein, 249 aa, Artificial Sequence
  Synthetic amino acid sequence Gelonin
GLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLFVDKDPK

SEQ ID NO: 3 - protein, 363 aa, Artificial Sequence
  Synthetic amino acid sequence C7rGel (anti-CEA Fn3-recombinant gelonin)
ISEFASVSDGTLSRDLGVVAATPTSLLISWYYSYSHHYSSYRITYGETGGNSPVQEFTVPRYRAFATISGLKPGVDYTITVYAVTSSSSYSYPISINYRTEIDKPSQGSGGGGSGLDTVSFSTKGATITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDNAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPK

SEQ ID NO: 4 - protein, 101 aa, Artificial Sequence
  Synthetic amino acid sequence Binding agent in SEQ ID NO 3
VSDGTLSRDLGVVAATPTSLLISWYYSYSHHYSSYRITYGETGGNSPVQEFTVPRYRAFATISGLKPGVDYTITVYAVTSSSSYSYPISINYRTEIDKPSQ

SEQ ID NO: 5 - protein, 7 aa, Artificial Sequence
  Synthetic amino acid sequence Linker in SEQ ID NO 3
GSGGGGS

SEQ ID NO: 6 - protein, 360 aa, Artificial Sequence
  Synthetic amino acid sequence E4rGel (anti-EGFR Fn3 recombinant gelonin)
ISEFASVSDVPRDLEVVAATPTSLLISWYHPFYYVAHSYRITYGETGGNSPVQEFTVPRSPWFATISGLKPGVDYTITVYAVTDSNGSHPISINYRTEIDKPSQGSGGGGSGLDTVSFSTKGATYITVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENADNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPK

SEQ ID NO: 7 - protein, 98 aa, Artificial Sequence
  Synthetic amino acid sequence Binding agent in SEQ ID NO 6
VSDVPRDLEVVAATPTSLLISWYHPFYYVAHSYRITYGETGGNSPVQEFTVPRSPWFATISGLKPGVDYTITVYAVTDSNGSHPISINYRTEIDKPSQ

SEQ ID NO: 8 - protein, 510 aa, Artificial Sequence
  Synthetic amino acid sequence 3ErGel (anti-CEA ds-scFv recombinant gelonin)
AMADIEFASQVKLEQSGAEVVKPGASVKLSCKASGFNIKDSYMHWLRQGPGQCLEWIGWIDPENGDTEYAPKFQGKATFTTDTSANTAYLGLSSLRPEDTAVYYCNEGTPTGPYYFDYWGQGTLVTVSGGGGSGGGGSGGGGSENVLTQSPSSMSVSVGDRVTIACSASSSVPYMHWLQQKPGKSPKLLIYLTSNLASGVPSRFSGSGSGTDYSLTISSVQPEDAATYYCQQRSSYPLTFGCGTKLEIKAAAGSGGGGSLQGLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSPSLEGEKAYRETTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKILLKFVDKDPK

SEQ ID NO: 9 - protein, 252 aa, Artificial Sequence
  Synthetic amino acid sequence Binding agent in SEQ ID NO 8
AMADIEFASQVKLEQSGAEVVKPGASVKLSCKASGFNIKDSYMHWLRQGPGQCLEWIGWIDPENGDTEYAPKFQGKATFTTDTSANTAYLGLSSLRPEDTAVYYCNEGTPTGPYYFDYWGQGTLVTVSGGGGSGGGGSGGGGSENVLTQSPSSMSVSVGDRVTIACSASSSVPYMHWLQQKPGKSPKLLIYLTSNLASGVPSRFSGSGSGTDYSLTISSVQPEDAATYYCQQRSSYPLTFGCGTKLEIKAAA

SEQ ID NO: 10 - protein, 508 aa, Artificial Sequence
  Synthetic amino acid sequence FErGel (anti-CEA ds-scFv recombinant gelonin)
AMADIEFASQVKLEQSGAEVVKPGASVKLSCKASGFNIKDSYMHWLRQGPGQCLEWIGWIDPENGDTEYAPKFQGKATFTTDTSANTAYLGLSSLRPEDTAVYYCNEGTPTGPYYFDYWGQGTLVTVSGGGGSGGGGSGGGGSENVLTQSPSSMSASVGDRVTIACSASSSVPYMHWFQQKPGKSPKLLIYSTSNLASGVPSRFSGSGSGTDYSLTISSVQPEDAATYYCQQRSSYPLTFGCGTKLEIKAAAGSGGGSGLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRTTDLGIEPLRIGIKKLDENAIDNYKPTEIASSLLVVIQMVSEAARFTFIENQIRNNFQQRIRPANNTISLENKWGKLSFQIRTSGANGMFSEAVELERANGKKYYVTAVDQVKPKIALLKFVDKDPK

SEQ ID NO: 11 - protein, 638 aa, Artificial Sequence
  Synthetic amino acid sequence C7LLO (anti-CEA Fn3 listeriolysin O)
ISEFASVSDGTLSRDLGVVAATPTSLLISWYYSYSHHYSSYRITYGETGGNSPVQEFTVPRYRAFATISGLKPGVDYTITVYAVTSSSSYSYPISINYRTEIDKPSQGSGGGGSKKIMLVFITLILVLPIAQQTEAKDASAFNKENLISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAIKNNSEYIETTSKAYTDGKINIDHSGGYVAQFNISWDEINYDPEGNEIVQHKNWSENNKSKLAHFTSSIYLPGNARNINVYAKECTGLAWEWWRTVIDDRNLPLVKNRNISIWGTTLYPKYSNSVDNPIE

SEQ ID NO: 12 - protein, 524 aa, Artificial Sequence
  Synthetic amino acid sequence LLO in SEQ ID NO 11
KKIMLVFITLILVLPIAQQTEAKDASAFNKENLISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAIKNNSEYIETTSKAYTDGKINIDHSGGYVAQFNISWDEINYDPEGNEIVQHKNWSENNKSKLAHFTSSIYLPGNARNINVYAKECTGLAWEWWRTVIDDRNLPLVKNRNISIWGTTLYPKYSNSVDNPIE

SEQ ID NO: 13 - protein, 636 aa, Artificial Sequence
  Synthetic amino acid sequence E6LLO (anti-EGFR Fn3 listeriolysin O)
ISEFASVSDVPRDLEVVAATPTSLLISWFDYAVTYYRITYGETGGNSPVQEFTVPGWISTATISGLKPGVDYTITVYAVTDNSRWPFRSTPISINYRTEIDKPSQGSGGGGSKKIMLVFITLILVSLIAQQTEAKDASAFNKENLISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVINNSEYIETTSKAYTDGKINIDHSGGYVAQFNISWDEINYDPEGNEIVQHKNWSENNKSKLAHFTSSIYLPGNARNINVYAKECTGLAWEWWRTVIDDRNLPLVKNRNISIWGTTLYPKYSNSVDNPIE

SEQ ID NO: 14 - protein, 589 aa, Artificial Sequence
  Synthetic amino acid sequence C7PFO (anti-CEA Fn3 perfringolysin O)
SASVSDGTLSRDLGVVAATPTSLLISWYYSYSHHYSSYRITYGETGGNSPVQEFTVPRYRAFATISGLKPGVDYTITVYAVTSSSSYSYPISINYRTEIDKPSQGSGGGGSSSKDITDKNQSIDSGISLSYNRNEVLASNGDKIESFVPKEGKKAGNKFIVVERQKRSLTTSPVDISIIDSVNDRTYPGALQLADKAFVENRPTILMVKRKPININIDLPGLKGENSIKVDDPTYGKVSGAIDELVSKWNEKYSTHTLPARTQYSESMVYSKSQISSALNVNAKVLENSLGVDFNAVANNEKKVMILAYKQIFYTVSADLPKNPSDLFDDSVTFNDLKQKGVSNEAPPLMVSNVAYGRTIYVKLETTSSSKDVQAAFKALINTDIKNSQQYKDIYENSSFTAVVLGGDAQEHNKVVTKDFDEIRKVIKDNATFSTKNPAYPISYTSVFLKDNSVAAVHNKTDYIETTSTEYSKGKINLDHSGAYVAQFEVAWDEVSYDKEGNEVLTHKWDGNYQDKTAHYSTVIPLEANARNIRIKAREATGLAWEWWRDVISEYDVPLTNNINVSIWGTTLYPGSSITYNGSHHHHHH

SEQ ID NO: 15 - protein, 470 aa, Artificial Sequence
  Synthetic amino acid sequence PFO in SEQ ID NOs 14 and 16
SSKDITDKNQSIDSGISLSYNRNEVLASNGDKIESFVPKEGKKAGNKFIVVERQKRSLTTSPVDISIIDSVNDRTYPGALQLADKAFVENRPTILMVKRKPININIDLPGLKGENSIKVDDPTYGKVSGAIDELVSKWNEKYSTHTLPARTQYSESMVYSKSQISSALNVNAKVLENSLGVDFNAVANNEKKVMILAYKQIFYTVSADLPKNPSDLFDDSVTFNDLKQKGVSNEAPPLMVSNVAYGRTIYVKLETTSSSKDVQAAFKALINTDIKNSQQYKDIYENSSFTAVVLGGDAQEHNKVVTKDFDEIRKVIKDNATFSTKNPAYPISYTSVFLKDNSVAAVHNKTDYIETTSTEYSKGKINLDHSGAYVAQFEVAWDEVSYDKEGNEVLTHKWDGNYQDKTAHYSTVIPLEANARNIRIKAREATGLAWEWWRDVISEYDVPLTNNINVSIWGTTLYPGSSITYN

SEQ ID NO: 16 - protein, 587 aa, Artificial Sequence
  Synthetic amino acid sequence E6PFO (anti-EGFR Fn3 perfringolysin O)
SASVSDVPRDLEVVAATPTSLLISWFDYAVTYYRITYGETGGNSPVQEFTVPGWISTATISGLKPGVDYTITVYAVTDNSRWPFRSTPISINYRTEIDKPSQGSGGGGSSSKDITDKNQSIDSGISSSYNRNEVLASNGDKIESFVPKEGKKAGNKFIVVERQKRSLTTSPVDISIIDSVNDRTYPGALQLADKAFVENRPTILMVKRKPININIDLPGLKGENSIKVDDPTYGKVSGAIDELVSKWNEKYSSTTLPARTQYSESMVYSKSQISSALNVNAKVLENSLGVDFNAVANNEKKVMILAYKQIFYTVSADLPKNPSDLFDDSVTFNDLKQKGVSNEAPPLMVSNVAYGRTIYVKLETTSSSKDVQAAFKALIKNDIKNSQQYKDIYENSSFTAVVLGGDAQEHNKVVTKDFDEIRKVIKDNATFSTKNPAYPISYTSVFLKDNSVAAVHNKTDYIETTSTEYSKGKINLDHSGAYVAQFEVAWDEVSYDKEGNEVLTHKTWGNYQDKTAHYSTVIPLEANARNIRIKAREATGLAWEWWRDVISEYDVPLTNNINVSIWGTTLYPGSSITYNGSHHHHHH

SEQ ID NO: 17 - protein, 6 aa, Artificial Sequence
  Synthetic amino acid sequence C-terminal His motif
HHHHHH

SEQ ID NO: 18 - protein, 8 aa, Artificial Sequence
  Synthetic amino acid sequence Strep-tag
WSHPQFEK

SEQ ID NO: 19 - protein, 30 aa, Artificial Sequence
  Synthetic amino acid sequence exemplary GALA peptide
WEAALAEALAEALAEHLAEALAEALEALAA

SEQ ID NO: 20 - protein, 34 aa, Artificial Sequence
  Synthetic amino acid sequence exemplary KALA peptide
WEAKLAKALAKALAKHLAKALAKALAKALKACEA

SEQ ID NO: 21 - protein, 20 aa, Artificial Sequence
  Synthetic JTS1 amino acid sequence
GLFEALLELLESLWELLLEA

SEQ ID NO: 22 - protein, 26 aa, Artificial Sequence
  Synthetic Melittin amino acid sequence
GIGAVLKVLTTGLPALISWIKRKRQQ

SEQ ID NO: 23 - protein, 4 aa, Artificial Sequence
  Synthetic amino acid sequence Repeated peptide motif for GALA peptides
EXLA

SEQ ID NO: 24 - protein, 4 aa, Artificial Sequence
  Synthetic amino acid sequence Repeat sequence in KALA peptides
KALA

(19) United States
(12) Patent Application Publication
(10) Pub. No.: US 2014/0080766 A1
(43) Pub. Date: Mar. 20, 2014

(54) COMPOSITIONS AND METHODS FOR MACROMOLECULAR DRUG DELIVERY

(71) Applicants: Christopher Michael Pirie, Cambridge, MA (US); David Victor Liu, San Francisco, CA (US); Nicole Jie Yeon Yang, Cambridge, MA (US); Karl Dane Wittrup, Chestnut Hill, MA (US)

(72) Inventors: Christopher Michael Pirie, Cambridge, MA (US); David Victor Liu, San Francisco, CA (US); Nicole Jie Yeon Yang, Cambridge, MA (US); Karl Dane Wittrup, Chestnut Hill, MA (US)

(73) Assignee: MASSACHUSETTS INSTITUTE OF TECHNOLOGY, Cambridge, MA (US)

(21) Appl. No.: 13/978,072

(22) PCT Filed: Jan. 7, 2012
(86) PCT No.: PCT/US2012/020567

§ 371 Date: Oct. 8, 2013

Related U.S. Application Data
(60) Provisional application No. 61/430,823, filed on Jan. 7, 2011.
(60) Provisional application No. 61/507,637, filed on Jul. 14, 2011.
(60) Provisional application No. 61/550,478, filed on Oct. 24, 2011.

(51) Int. Cl. A61K 47/48; A61K 31/713

(52) U.S. Cl. CPC A61K 47/48246; A61K 31/713
USPC 514/20.9; 530/350; 536/23.4; 435/320.1; 435/252.33; 530/358

(57) ABSTRACT

The present invention features compositions and methods for delivering a therapeutic agent to the cytoplasm of a cell. We have developed, inter alia, a system in which two or more distinct moieties—at least one therapeutic moiety and at least one potentiating moiety—selectively target and specifically bind cell surface molecules that are then internalized to an intracellular, membrane-bound compartment, such as an endosome. In some embodiments, as discussed further below, a third moiety that induces clustering of the targeted cell surface molecule can also be employed. Regardless of whether the compositions and methods include two or three moieties, the therapeutic agent can be any agent one wishes to deliver to the cytoplasm of a cell, and the potentiating agent can be any agent that destabilizes the intracellular, sub-cellular compartment in which the therapeutic agent is sequestered. The potentiating moiety can include, for example, a lytic agent (i.e., an agent that lyses or otherwise increases the permeability of the membrane of the intracellular compartment containing the therapeutic agent). To direct the various moieties of the system, including the therapeutic, potentiating, and clustering moieties, to a selected cellular target, any of the moieties can include a binding agent that selectively targets and specifically binds a molecule present on the surface of the targeted cell.

### BACKGROUND

In the biotechnology industry, there is a great interest in being able to deliver macromolecules such as DNA, RNA, and proteins to the cytoplasm of cells in order to affect intracellular targets. The utility of any biomacromolecule in vivo is fraught with numerous challenges, not the least of which is endosomal escape (Shim et al. FEBS J 277:4814-4827, 2010). Researchers working in the fields of gene therapy and RNA interference understand the importance of achieving intracellular delivery and the value of accomplishing delivery in a targeted manner (see, for example, Lares et al., Trends Biotechnol. 28:570-579, 2010; and Varkouhi et al., J. Controlled Release 151:220-228, 2011). Delivery of protein antigens to antigen presenting cells to elicit an immune response is also enhanced by various mechanisms of endosomal disruption (see, e.g., Paterson et al., Semin. Immunol. 22:183-189, 2010; and Foster et al., Bioconjug. Chem. 21:2205-2212, 2010).

In order to be effective, immunotoxins must be delivered in a targeted manner and reach the cytoplasm of the cells to which they are delivered. However, there has been diminished interest in the problems associated with delivery of these proteins, partly for historical reasons. The early developers of immunotoxins diverged in their use of different types of toxins, generally working with either Type I or Type II ribosome inactivating proteins. The researchers working with Type II toxins, such as diphtheria toxin, ricin, or pseudomonas exotoxin, were not concerned about intracellular delivery because these toxins incorporate their own evolved translocation domains that facilitate endosomal escape (Kelley et al., Proc. Natl. Acad. Sci. USA 85:3980-3984, 1988; Bjorn et al., Cancer Res. 46:3262-3267, 1986; Seon, Cancer Res. 44:259-264, 1984). Others, usually those working with Type I toxins, investigated various small molecule and protein-based methods for enhancing translocation with moderate success in vitro (see, for example, Wu, Br. J. Cancer 75:1347-1355, 1997; Wu et al., J. Cell Biol. 125:743-753, 1994; Vitetta et al., Proc. Natl. Acad. Sci. USA 80:6332-6335, 1983; Greenfield et al., Science 238:536-539, 1987; Goldmacher et al., Mol. Pharmacol. 36:818-822, 1989; and FitzGerald et al., Proc. Natl. Acad. Sci. USA 80:4134-4138, 1983). In some cases, potent intracellular delivery agents have been identified, but specificity remains elusive and toxicity is often a confounding factor.

### SUMMARY

The present invention features compositions and methods for delivering a therapeutic agent to the cytoplasm of a cell. We have developed, inter alia, a system in which two or more distinct moieties—at least one therapeutic moiety and at least one potentiating moiety—selectively target and specifically bind cell surface molecules that are then internalized to an intracellular, membrane-bound compartment, such as an endosome. In some embodiments, as discussed further below, a third moiety that induces clustering of the targeted cell surface molecule can also be employed. Regardless of whether the compositions and methods include two or three moieties, the therapeutic agent can be any agent one wishes to deliver to the cytoplasm of a cell, and the potentiating agent can be any agent that destabilizes the intracellular, sub-cellular compartment in which the therapeutic agent is sequestered. The potentiating moiety can include, for example, a lytic agent (i.e., an agent that lyses or otherwise increases the permeability of the membrane of the intracellular compartment containing the therapeutic agent). To direct the various moieties of the system, including the therapeutic, potentiating, and clustering moieties, to a selected cellular target, any of the moieties can include a binding agent that selectively targets and specifically binds a molecule present on the surface of the targeted cell.

Among the compositions of the invention are: (a) a therapeutic moiety including a binding agent and a therapeutic agent; (b) a potentiating moiety including a binding agent and a lytic agent; (c) a clustering moiety including a plurality of binding agents that bind more than one distinct epitope on a cell surface molecule; (d) a therapeutic agent that is free from (i.e., not bound or otherwise joined to) a binding agent; (e) a potentiated therapeutic moiety including a therapeutic agent, a binding agent, and a lytic agent; (f) a cluster-inducing therapeutic moiety including a therapeutic agent and a plurality of binding agents that bind more than one distinct epitope on a cell surface molecule; (g) a cluster-inducing potentiating moiety including a lytic agent and a plurality of binding agents that bind more than one distinct epitope on a cell surface molecule; and (h) a cluster-inducing therapeutic moiety further including a lytic agent. The latter moiety includes a therapeutic agent, a lytic agent, and a plurality of binding agents that bind more than one distinct epitope on a cell surface molecule.

We may sometimes refer to the agents described as (a)-(h) in the preceding paragraph as Agents A-H, respectively (e.g., we may refer to the therapeutic moiety as Agent A). For the sake of added clarity, we note that when a moiety includes two or more agents it may consist of those agents or comprise those agents. Similarly, for the sake of added clarity, we note that a therapeutic agent that is free from (i.e., not bound or otherwise joined to) a binding agent is not a novel composition but is suitable for inclusion in the novel compositions and methods of the invention. It is to be understood that the moieties of the present invention are not naturally occurring in their entirety but may include sequences or component parts (e.g., proteins, nucleic acids, small molecules, or polymers) that are naturally occurring (e.g., that are produced by biological cells in a natural setting). As indicated above, various functional attributes important to the present system can be divided among individual moieties (e.g., a therapeutic agent and a lytic agent can be incorporated into separate moieities (Agents A and B, respectively) or consolidated (e.g., a therapeutic agent and a lytic agent can be incorporated into the same moiety (Agent E)). Similarly, either the therapeutic moiety or the potentiating moiety can be engineered to include a plurality of binding agents (Agents F and G, respectively). Where one or both of those moieties are multi-specific, a separate clustering moiety may not be required (as the multi-specific binding agent(s) on the therapeutic and/or potentiating moieties would carry out the clustering effect otherwise mediated by the clustering moiety).

While a therapeutic agent (e.g., a macromolecule that is active intracellularly) can be a part of a larger moiety, the invention is not so limited. Thus, in another aspect, a therapeutic agent can be delivered without being fused or otherwise joined to a binding agent (i.e., the therapeutic agent can be free of a larger moiety that includes a binding agent). For example, a potentiating moiety, as described herein, and, optionally, a clustering moiety, can be administered with essentially any “free” therapeutic agent to facilitate the internalization of the therapeutic agent and its release from an intracellular compartment (where sequestration inhibits the desired therapeutic effect on a cell, tissue, or patient to which (or whom) the therapeutic has been administered). Accordingly, in one aspect, the invention features a pharmaceutical composition comprising a therapeutic agent that is not bound to a binding agent and at least one other agent (e.g., a potentiating and/or complexing moiety (i.e., Agents B and/or C) or a moiety that incorporates both a lytic agent and a plurality of binding agents (i.e., Agent G)). In another aspect, the invention features compositions (e.g., a pharmaceutical composition) including a therapeutic moiety (which includes a binding agent) and a potentiating and/or complexing moiety or a moiety that incorporates both a lytic agent and a plurality of binding agents (i.e., Agent G). Another composition of the invention is a kit in which various moieties are packaged together in separate containers (i.e., the kits include a plurality of containers within a single overall packaged unit). Because the studies we have conducted to date indicate there may be advantages to administering the moieties separately, various moieties (e.g., Agents A-C, B-D or D and G) can be placed in separate containers for administration at different times and/or by different routes of administration.

The same binding agent can be incorporated into more than one type of moiety. For example, a binding agent that is a single chain antibody (scFv) or modified fibronectin domain that binds an epidermal growth factor receptor can be incorporated into either a therapeutic moiety or a potentiating moiety; the same agent may be incorporated in either case. However, it may be preferable when administering the therapeutic moiety and the potentiating moiety to select a therapeutic moiety that has one binding agent and a potentiating moiety that has another binding agent. For example, as illustrated in FIG. 1, a targeted biotherapeutic (Agent A) can specifically bind Antigen 1 and a targeted lysin (Agent B) can specifically bind Antigen 2. It will be apparent to one of ordinary skill in the art that there is considerable potential to vary the individual components incorporated into the moieties described herein. For example, the system illustrated in FIG. 1 can be readily modified so Agent A binds Antigen 2 and Agent B binds Antigen 1.

The compositions of the invention encompass the moieties described above, nucleic acids that encode them (or an agent within them), expression vectors and host cells that contain those nucleic acids, pharmaceutical formulations, and kits (e.g., kits in which the therapeutic and potentiating agents are combined or contained in separate containers together with instructions for use). Also within the scope of the present invention are methods of making and using the compositions described above and elsewhere herein. Any of the methods of treatment described herein can be alternatively presented as methods of use. Accordingly, the invention features the use of a moiety described herein (or any combination of moieties or components as described herein) for the preparation of a medicament. The invention features the use of a moiety described herein (or any combination of moieties or components as described herein) for the preparation of a medicament for the treatment of cancer, autoimmune disease, or infectious disease.

The invention is exemplified with certain constructs. We have engineered the 10^{th} Type III human fibronectin domain (Fn3) to specifically bind to an EGFR or carcinoembryonic antigen (CEA), and we have fused such binding agents to listeriolysin O (LLO). In our particular studies to date, the modified Fn3 domain that binds CEA was linked to a therapeutic agent, and the modified Fn3 domain that binds the EGFR was linked to LLO. In this example, the former fusion protein serves as the therapeutic agent and the latter fusion protein serves as the potentiating agent. It will be apparent from the description herein that the moieties can be configured differently and that other proteins can be used instead of a fibronectin domain. Binding agents, including immunoglobulin and immunoglobulin-like molecules (e.g., IgGs, scFvs, Fab′ fragments, F(ab′)2 fragments and antibody mimics (e.g., monobodies) as well as non-immunoglobulin-like molecules (e.g., modified or engineered fibronectin domains and ankyrin repeat-based proteins) are well known and understood in the art.

Where the moiety is a fusion protein, it can be expressed recombinantly in expression systems, such as E. coli. Our fusion proteins conserved specific binding and showed some antigen-dependent cytotoxicity. pH dependent hemolysis, a characteristic of listeriolysin O, was also conserved. When combined in cell treatments with immunotoxins based on the plant toxin gelonin, these fusions exhibited potentiating activity, decreasing the IC₅₀ values of the immunotoxins by three orders of magnitude. Potentiation was observed even when the two agents were targeted to the same antigen competitively, when targeted to different antigens known to colocalize, or when treating cells expressing low levels of antigens. Such strategies and cells are encompassed by the present invention. We went on to test the ability of the fusions to potentiate immunotoxin activity even when the two agents were applied to cells at different times and to quantify the reduction in the intracellular barrier to delivery, which was consistent with the improvement in cytotoxicity, suggesting that the observed synergistic effects are the result of membrane destabilization or pore-formation by the lysin leading to release of immunotoxin. The immunotoxins we tested showed enhanced, targeted cytotoxicity towards highly-expressing antigen positive cells. However, they were impotent against lower-expressing cell lines, which led us to investigate and quantify the endosomal escape efficiency of the immunotoxins. Our findings suggested that, on average, millions of immunotoxin molecules had to be internalized by a single cell before the handful of toxins necessary to induce apoptosis in the cell would reach the cytoplasm. This result encouraged us design tools capable of inducing targeted endosomal disruption.

Accordingly, in one aspect, the invention features a potentiating moiety including (a) a binding agent that specifically binds a cell surface molecule, (b) a lytic agent that destabilizes the membrane of an intracellular compartment and (c) optionally, a linker between the binding agent and the lytic agent. The binding agent can be a tenth Type III fibronectin domain engineered to specifically bind the cell surface molecule one wishes to target, and the lytic agent can be listeriolysin O, an isoform thereof, a homolog thereof, or a biologically active fragment or other variant thereof. The cell surface molecule can be a tyrosine kinase receptor or a tumor antigen (i.e. a molecule that is selectively expressed (e.g., overexpressed or misexpressed) by a tumor cell). The binding agent and the lytic agent can be joined as a fusion protein, a chemical conjugate, or in a non-covalent complex. Any of the potentiating moieties can further include a therapeutic agent (e.g., a macromolecule as described herein). The potentiating moiety can also be configured to include a plurality of binding agents that bind more than one distinct epitope on a cell surface molecule, such that the moiety as a whole is multi-specific and constitutes a cluster-inducing therapeutic moiety further including a lytic agent.

In another aspect, the invention features a cluster-inducing therapeutic moiety that includes a therapeutic agent and a plurality of binding agents that bind more than one distinct epitope on a cell surface molecule. In another aspect, the invention features a cluster-inducing potentiating moiety that includes a lytic agent and a plurality of binding agents that bind more than one distinct epitope on a cell surface molecule. Any of these moieties can be configured as a fusion protein or a chemical conjugate. Any of the agents within these moieties can be joined in a non-covalent complex.

In another aspect, the invention features nucleic acids that include a sequence that encodes the fusion proteins described herein or an agent suitable for inclusion in a moiety. For example, the nucleic acid can encode a fusion protein that includes, from the N-terminus to the C-terminus: maltose binding protein, N₁₀ linker, a Factor Xa protease site, the tenth Type III fibronectin domain, a G₄S linker, and listeriolysin O or an isoform, homolog, biologically active fragment or other variant thereof.

In another aspect, the invention features expression vectors that include the nucleic acids described herein and/or an expression vector (e.g., a plasmid).

In another aspect, the invention features host cells that include one or more of the nucleic acids and/or expression vectors described herein. The field of molecular biology has advanced such that expression vector and host cells are well known and understood in the art. Host cells useful in the present invention can be prokaryotic cells (such as the bacterial cell E. coli).

In another aspect, the invention features kits that include one or more of the potentiating moieties described herein; the cluster-inducing therapeutic moieties described herein; tthe cluster-inducing potentiating moieties described herein; the nucleic acids described herein; the expression vectors described herein; a host cell as described herein; and instructions for use.

In another aspect, the invention features compositions, including pharmaceutical compositions and lyophilized or otherwise preserved or formulated moieties as described herein. These compositions can also include the nucleic acids, expression vectors, and host cells of the invention.

In another aspect, the invention features methods of delivering a therapeutic agent to a cell. These methods can be carried out by administering to a patient in need of the therapeutic agent a therapeutically effect amount of: (a) a potentiating moiety as described herein and (b) a therapeutic moiety or therapeutic agent (i.e., a therapeutic agent that is free of a binding agent). Given the teaching of the invention, it will be clear that other moieties and combinations of moieties and therapeutc agents can also be administered. The patient may have cancer and the binding agent in that case can be one that specifically binds a cancer cell-associated antigen (i.e., an antigen expressed by the cancer cells within the patient) and the therapeutic agent can be a chemotherapeutic agent.

As noted, the therapeutic methods of the invention can be described in terms of use. Accordingly, the invention features the use of a potentiating moiety, a cluster-inducing therapeutic moiety, a cluster-inducing potentiating moiety, a nucleic acid, an expression vector, and/or a host cell as described herein in the preparation of a medicament (e.g., a medicament for the treatment of cancer).

The development of various proteins that can specifically bind a given target has allowed for improved tissue-specific intervention, but the use of these proteins in the field of intracellular drug delivery has been limited. As a result, there is still a need for a targeted intracellular delivery system, particularly if that system were broadly applicable. We have validated the independently targeted drug delivery system described herein using gelonin immunotoxins whose exquisite cytoplasmic activity make them ideal candidates for displaying effects of enhanced intracellular delivery. Our studies conducted both in vitro and in vivo demonstrate the ability of the fusion proteins exemplified below to synergize in both antigen-specific cytotoxicity and tumor growth inhibition, reducing the number of internalized toxins required to induce apoptosis from ˜5×10⁶ to <10⁴ and significantly delaying tumor progression. These results convey both the potential of this particular application in cancer therapy and of our general approach to intracellular delivery of therapeutic macromolecules.

### BRIEF DESCRIPTION OF THE DRAWINGS

FIG. 1 is an illustration of a two-agent intracellular delivery system in keeping with the invention. Without a potentiating moiety, intracellularly active therapeutics are directed to endosomes before degradation in lysosomes (A). However, upon compartmental colocalization of a therapeutic moiety and a potentiating moiety, the lytic agent in the potentiating moiety destabilizes the endosome and the therapeutic agent is released to the cytoplasm rather than degraded in a lysosome (B).

FIG. 2 is a tri-panel fluorescentmicrograph showing colocalization of intracellular EGFR and CEA. HT-29 cells that express both EGFR and CEA demonstrate that agents targeted to these two receptors will colocalize to a considerable extent to the same intracellular compartments, which we believe to be necessary for the potentiation strategy described herein. An anti-EGFR antibody was labeled with AlexaFluor-488 and an anti-CEA scFv was labeled with AlexaFluor-594 before both were used to label HT-29 cells to observe colocalization.

FIG. 3 is a graph depicting internalized cytotoxicity data from A431 and HT-29 cells treated with a CEA targeted gelonin toxin fusion (C7rGel) and an EGFR targeted gelonin toxin fusion (E4rGel), respectively, in conditions with or without a potentiating moiety. In the presence of a potentiator, significantly less immunotoxin uptake is required to induce loss of viability.

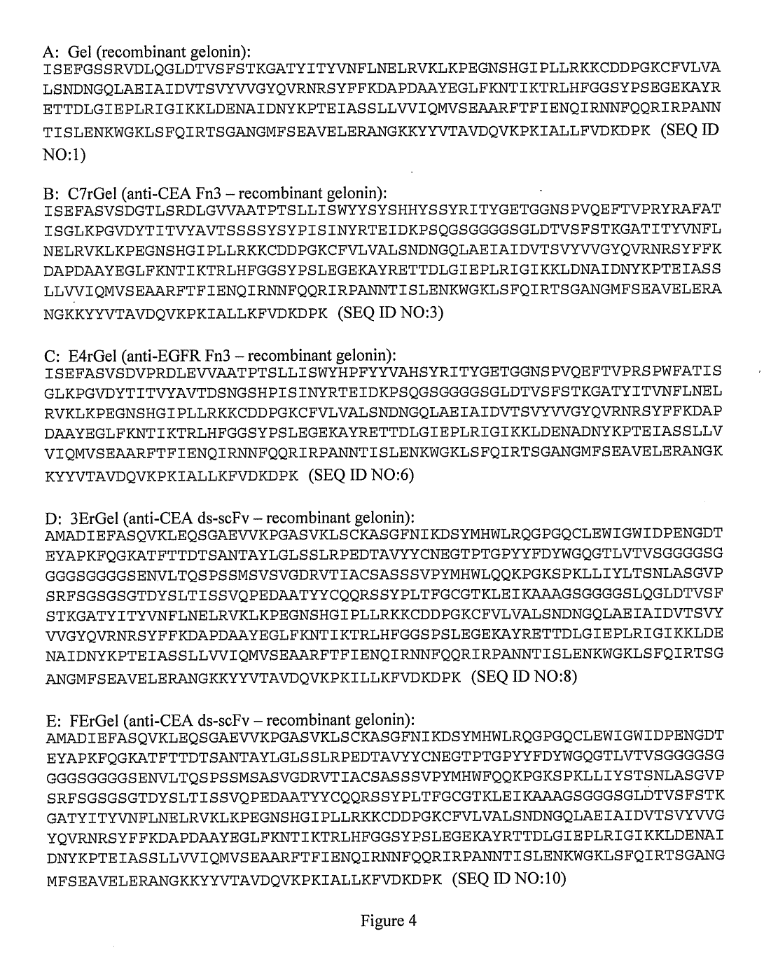

FIG. 4 is a series of amino acid sequences designated “A” through “I”. Sequence A (SEQ ID NO:1) comprises a recombinant gelonin construct with the toxin extending from residue 13 to residue 261 (SEQ ID NO:2). Sequence B (SEQ ID NO:3) represents a fusion protein in which a binding agent (a fibronectin type 3 domain engineered to bind CEA) is fused to a therapeutic agent (the toxin gelonin). The binding agent extends from residue 7 to residue 107 (SEQ ID NO:4). A linker from residue 108 to residue 114 links the binding agent to the therapeutic agent (SEQ ID NO:5). Sequence C (SEQ ID NO:6) represents a fusion protein in which a binding agent (a fibronectin type 3 domain engineered to bind an EGFR) is fused to a therapeutic agent (the toxin gelonin). The binding agent extends from residue 7 to residue 104 (SEQ ID NO:7). A linker from residue 105 to residue 111 links the binding agent to the therapeutic agent (SEQ ID NO:5). Sequence D (SEQ ID NO:8) represents a fusion protein in which a binding agent (a disulfide stabilized single chain immunoglobulin variable fragment engineered to bind CEA) is fused to a therapeutic agent (the toxin gelonin). The binding agent extends from residue 10 to residue 252 (SEQ ID NO:9). A linker from residue 253 to residue 259 links the binding agent to the therapeutic agent (SEQ ID NO:5). The gelonin sequence completes the C-terminal domain (SEQ ID NO:2, which sequence may optionally include the 2-mer LQ at the N-terminal end of the fusion protein (as shown in Sequence D). Sequence E (SEQ ID NO:10) represents a fusion protein in which a binding agent (a disulfide stabilized single chain immunoglobulin variable fragment engineered to bind CEA) is fused to a therapeutic agent (the toxin gelonin). The binding agent extends from residue 10 to residue 252 (SEQ ID NO:9). A linker from residue 253 to residue 259 links the binding agent to the therapeutic agent (SEQ ID NO:5). Gelonin completes the C-terminal sequence. Sequence F (SEQ ID NO:11) represents a fusion protein in which a binding agent (a fibronectin type 3 domain engineered to bind CEA) is fused to a potentiating agent (the cytolysin listeriolysin O). The binding agent extends from residue 7 to residue 107 (SEQ ID NO:4). A linker from residue 108 to 114 links the binding agent to the potentiating agent (SEQ ID NO:5). The lytic agent LLO completes the C-terminus of the fusion protein (SEQ ID NO:12). Sequence G (SEQ ID NO:13) represents a fusion protein in which a binding agent (a fibronectin type 3 domain engineered to bind EGFR) is fused to a potentiating agent (the cytolysin listeriolysin O). The binding agent extends from residue 7 to residue 104 (SEQ ID NO:7). A linker from residue 105 to 111 links the binding agent to the potentiating agent (SEQ ID NO:5). The lytic agent LLO completes the C-terminus of the fusion protein (SEQ ID NO:12). Sequence H (SEQ ID NO:14) represents a fusion protein in which a binding agent (a fibronectin type 3 domain engineered to bind CEA) is fused to a potentiating agent (the cytolysin perfringolysin O). The binding agent extends from residue 7 to residue 107 (SEQ ID NO:4). A linker from residue 108 to 114 links the binding agent to the potentiating agent (SEQ ID NO:5). The lytic agent PFO completes the C-terminus of the fusion protein (SEQ ID NO:15). Sequence I (SEQ ID NO:16) represents a fusion protein in which a binding agent (a fibronectin type 3 domain engineered to bind EGFR) is fused to a potentiating agent (the cytolysin perfringolysin O). The binding agent extends from residue 7 to residue 104 (SEQ ID NO:7). A linker from residue 105 to 111 links the binding agent to the potentiating agent (SEQ ID NO:5). The lytic agent PFO (SEQ ID NO:15) completes the C-terminus of the fusion proteins. Sequences H and I also contain a C-terminal HHHHHH (SEQ ID NO:17) motif for metal affinity chromatography purification and immunological affinity tagging.

FIG. 5 illustrates various chemical reactions that can be carried out to form chemical conjugates including the agents described herein.

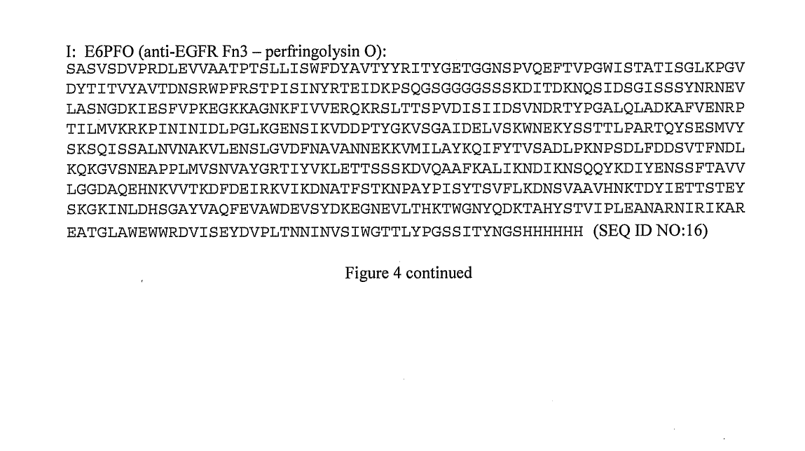

FIG. 6 is a schematic illustration of the protein constructs used in the work described in Example 3. E6N2 is a homodimeric fusion protein containing the dsRBD from human protein kinase R, the mouse IgG2a Fc fragment, and the EGFR-binding Fn3 clone, E6. D-PFO is a fusion protein with EGFR-binding Fn3 clone D, and the cholesterol dependent cytolysin, PFO. HNB-LCD is a multispecific construct containing EGFR-binding Fn3 clones B and D on the N terminus of the heavy chain and the C terminus of the light chain of cetixumab, respectively. All proteins are drawn with the N-terminus on top and the C-terminus on the bottom.

FIG. 7 is a line graph illustrating the results of an in vivo combination therapy. HT-29 tumor xenograft growth is inhibited slightly by independent drug treatments, but only the combination exhibits a significant delay in tumor growth (see Example 2). Arrows indicate days when doses were administered to all groups.

### DETAILED DESCRIPTION

Biotherapeutics have revolutionized medicine with their ability to achieve unprecedented molecular recognition and mediate complex biological responses. The intracellular delivery of biotherapeutics is an unmet scientific challenge and medical need. A wide variety of different treatment modalities depend on not only the ability to achieve intracellular delivery, but also on the ability to do so in a targeted manner.

In the paragraphs that follow, we describe in further detail the moieties useful in the present system, the agents that can be included in those moieties, and other features of the invention (e.g., compositions such as pharmaceutical compositions and kits). This description is provided as a non-limiting illustration of the invention. As noted, any of the moieties described herein as a therapeutic moiety (Agent A), a potentiating moiety (Agent B), a clustering moiety (Agent C), a potentiated therapeutic moiety (Agent E), a cluster-inducing therapeutic moiety (Agent F), a cluster-inducing potentiating moiety (Agent G), and a cluster-inducing therapeutic moiety (Agent H) can include a binding moiety and, as described herein, can include a plurality of binding agents that bind more than one distinct epitope on a cell surface molecule and render the moiety multi-specific. Accordingly, we begin with a further discussion of binding agents.

Binding Agents:

A binding agent is a suitable component of any of the moieties described herein (potentiating, therapeutic, or clustering) that mediates the association between the moiety and a surface molecule on the target cell that is subsequently internalized. The binding agent may be an immunoglobulin or an immunoglobulin-like molecule or it may be a non-immunoglobulin molecule, such as an engineered domain of fibronectin. Alternatively, the binding agent may be a native growth factor, signaling molecule, or cytokine.

Where the binding agent is an immunoglobulin or an immunoglobulin-like molecule (e.g., an scFv, an Fab, an F(ab′)2 fragment, a diabody, or a triabody), it can be an IgG of any subtype (e.g., an IgG1, IgG2, IgG3, or IgG4), and any of these subtypes can be chimeric, mammalian (e.g., human or murine) or humanized immunoglobulins. More specifically, the immunoglobulin can be cetuximab, panitumumab, trastuzumab, matuzumab (formerly EMD7000), h-R3 (TheraCIM® hR3; J. Clin. Oncol., May 2004) or the monoclonal antibody 806; a single chain antibody comprising the variable heavy and light chains of one of these antibodies; or an immunoglobulin that specifically binds the same target as one of these antibodies. Thus, a binding agent can be or can include a biologically active fragment or other variant of a commercially developed antibody or any antibody that specifically binds a molecular target as described herein (e.g., an EGFR). In some embodiments, a biologically active fragment or other variant of an immunoglobulin can include one or more of the CDRs, framework regions, or paratopes of a commercially developed antibody or any other known antibody that specifically binds a molecular target as described herein. For example, a first binding agent can specifically bind an epitope of a full-length, wild-type EGFR, and a second binding agent can specifically bind an epitope of a mutant (e.g., a truncation mutant) of the EGFR (e.g., EGFRvIII). The same is true for other receptor tyrosine kinases; the epitopes can differ by virtue of being present in a wild-type form of the molecular target and absent in a mutant form. Where a domain is an immunoglobulin-based polypeptide, it can include a variable domain that recognizes and specifically binds a cryptic epitope on the target receptor that is not exposed under native folding conditions.

Where the binding agent is a non-immunoglobulin molecule, it can be, but is not limited to, one or more of the following types of domains. By methods known and used in the art, any of these domains can be modified to specifically bind a given epitope: a lipocalin-based polypeptide; a ubiquitin-based polypeptides; a transferrin-based polypeptide; a protein A domain-based polypeptide; an ankyrin repeat-based polypeptide; a tetranectin-based polypeptide; a cysteine-rich domain-based polypeptide; a Fyn SH3 domain-based polypeptide; an EGFR A domain-based polypeptide; a centyrin-based polypeptide; and a Kunitz domain-based polypeptide.

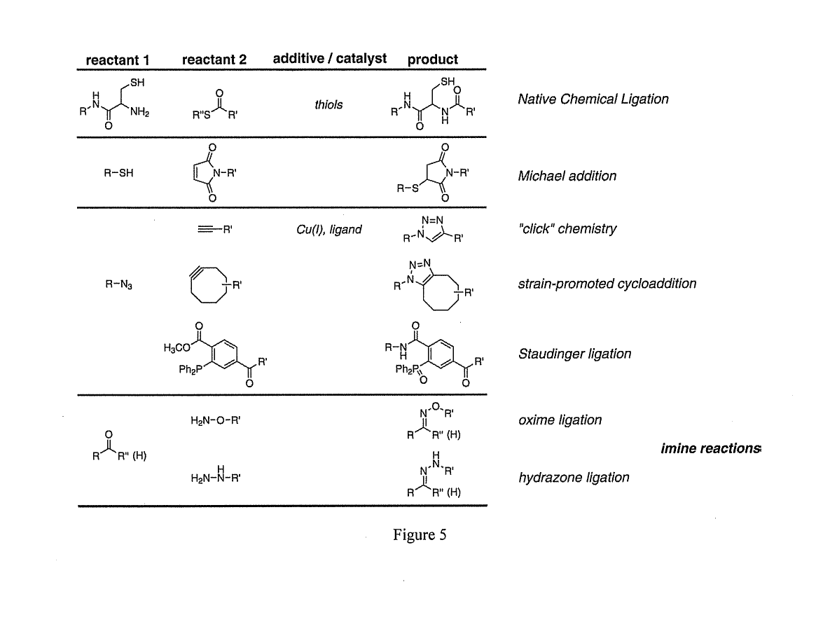

The moieties can differ with respect to the total number of binding agents they include. In some embodiments, a moiety (e.g., a potentiating moiety) may include a single binding domain. In some embodiments, a moiety (e.g., a therapeutic moiety) may include two binding domains (e.g., see the therapeutic moiety illustrated in FIG. 6). In some embodiments, a moiety (e.g., a clustering moiety) may include more than two (e.g., 3, 4, 5, or 6) binding domains (e.g., see the clustering moiety illustrated in FIG. 6). Further, as noted, the clustering functionality of the clustering moiety can be consolidated with one or more of the functionalities of the binding and/or potentiating domains by including multispecific binding domains in the moieties that also include a therapeutic agent or a lytic agent.

The moieties can differ with respect to the total number of binding sites they include (their valency), the number of different epitopes they bind (their specificity), and the number of different paratopes they include. To illustrate this terminology, a conventional monoclonal antibody is bivalent, monospecific, and monoparatopic. As noted, a clustering moiety (or a moiety designed to have clustering abilities) can be multivalent. When they include two binding sites, they are bivalent; where they include three binding sites, they are trivalent; when they include four binding sites, they are tetravalent; when they include six binding sites, they are hexavalent; when they include eight binding sites, they are octavalent, and so forth. While the constructs can be engineered to bind the same epitope on a molecular target (i.e., they can be monospecific), our current expectation is that constructs engineered to bind different epitopes on the same molecular target (multispecific constructs) will have superior efficacy. Accordingly, the moieties of the invention can specifically bind two distinct epitopes, making them “bispecific”; three distinct epitopes, making them “trispecific”; four distinct epitopes, making them “tetraspecific”; and so forth. Similarly, the binding domains can be engineered to include the same paratope (in which case they would be “monoparatopic”). The same paratope can be incorporated into different surrounding scaffolds. Thus, more than one type of domain (e.g., an immunoglobulin-like domain and a non-immunoglobulin-like domain) can include the same paratope.

Another way the engineered proteins of the present invention can be characterized is by their affinity for the molecular target they are designed to specifically bind. For example, one or more of the domains in an engineered protein construct may bind a molecular target with an affinity in the pM to nM range (e.g., an affinity of less than or about 1 pM, 10 pM, 25 pM, 50 pM, 100 pM, 250 pM, 500 pM, 1 nM, 5 nM, 10 nM, 15 nM, 20 nM, 25 nM, 30 nM, 40 nM or 50 nM). In addition to this characteristic, any given binding agent can be characterized in the context of the present systems in terms of its ability (or the combined abilities of the included moieties) to modify cell behavior (e.g., cellular proliferation or migration) or to positively impact a symptom of a disease, disorder, condition, syndrome, or the like, associated with the expression or activity of the molecular target. In vitro assays for assessing binding to a molecular target, cellular proliferation, and cellular migration are known in the art. For example, where the molecular target is an EGFR, binding, proliferation, and migration assays can be carried out using A431 epidermoid carcinoma cells, HeLa cervical carcinoma cells, and/or HT29 colorectal carcinoma cells. Other useful cells and cell lines will be known to those of ordinary skill in the art. For example, an engineered protein can be analyzed using U87 glioblastoma cells, hMEC cells (human mammary epithelial cells), or Chinese hamster ovary (CHO) cells. The molecular target can be expressed as a fluorescently tagged protein to facilitate analysis of an engineered protein's effect on the target. For example, the assays of the present invention can be carried out using a cell type as described above transfected with a construct expressing a tagged molecular target (e.g., an EGFR-green fluorescent protein fusion). With respect to the assayed parameter, one can measure receptor internalization or a downstream event such as cellular proliferation or migration (where the therapeutic agent mediates RNAi, one can measure expression of the targeted gene). Upon binding, an engineered protein may inhibit the measured parameter (e.g., cellular proliferation or migration (or gene expression)) by at least or about 30% (e.g., by at least or about 35%, 40%, 50%, 65%, 75%, 85%, 90%, 95% or more) relative to a control (e.g., relative to proliferation or migration in the absence of the antibody or a scrambled engineered protein).

One can also subject a binding agent of a moiety described herein to directed evolution in order to generate a modified variant with improved specificity and affinity for a given molecular target. Whether modified by directed evolution, random mutagenesis, or in some other manner, one or more of the amino acid residues in a biologically active variant of an agent (e.g., a peptide binding agent, peptide therapeutic, or lytic peptide) may be a non-naturally occurring amino acid residue. Naturally occurring amino acid residues include those naturally encoded by the genetic code as well as non-standard amino acids (e.g., amino acids having the D-configuration instead of the L-configuration). Peptides included in the present moieties can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine(2R,3S)-2-amino-3-methylpentanoic acid and L-cyclopentyl glycine (S)-2-amino-2-cyclopentyl acetic acid. For other examples, one can consult textbooks or the worldwide web (a site is currently maintained by the California Institute of Technology and displays structures of non-natural amino acids that have been successfully incorporated into functional proteins). Non-natural amino acid residues and amino acid derivatives listed in U.S. Application No. 20040204561 (see ¶ 0042, for example) can also be used.

Alternatively, or in addition, one or more of the amino acid residues in a biologically active variant can be a naturally occurring residue that differs from the naturally occurring residue found in the corresponding position in a wild type sequence. In other words, biologically active variants can include one or more amino acid substitutions. We may refer to a substitution, addition, or deletion of amino acid residues as a mutation of the wild type sequence. As noted, the substitution can replace a naturally occurring amino acid residue with a non-naturally occurring residue or just a different naturally occurring residue. Further the substitution can constitute a conservative or non-conservative substitution. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

The polypeptides that are biologically active variants of binding agent polypeptide or a lytic agent polypeptide can be characterized in terms of the extent to which their sequence is similar to or identical to the corresponding wild-type polypeptide. For example, the sequence of a biologically active variant can be at least or about 80% identical to corresponding residues in the wild type polypeptide. For example, a biologically active variant of a binding agent polypeptide or a lytic peptide can have an amino acid sequence with at least or about 80% sequence identity (e.g., at least or about 85%, 90%, 95%, 97%, 98%, or 99% sequence identity) to binding agent polypeptide or a lytic agent polypeptide or to a homolog or ortholog thereof. Methods for aligning amino acid sequences and nucleic acid sequences and determining % identity are well known in the art.

A biologically active variant of a peptide binding agent a lytic peptide will retain sufficient biological activity to be useful in the present methods. Their biological activity can be assessed in ways known to one of ordinary skill in the art and these include, without limitation, in vitro binding and competition assays, blockade of signal transduction, or cytotoxicity assays.

Lytic Agents:

One portion (e.g., a “first” portion) of a potentiating moiety can be a lytic agent that destabilizes the membrane of an intracellular compartment enough to allow the contents of the compartment to enter the cytoplasm. The lytic agent, whether proteinaceous or non-proteinaceous, can be one that is naturally occurring or non-naturally occurring. As with other agents incorporated in the moieties described herein, where a naturally occurring or known lysin (e.g., a commercially available polymer) is effective, a fragment or other variant thereof that has sufficient activity to allow the release of a sequestered therapeutic from an intracellular compartment will also be effective and can be incorporated in the compositions described herein.

The lytic agent can be a polypeptide. Useful polypeptides can be derived from either prokaryotic or eukaryotic sources. For example, the lytic agent can be a microbial cytolysin, for example a thiol-activated cytolysin such as listeriolysin O (LLO), ivanolysin, seeligeriolysin, perfringolysin O, streptolysin O, pneumolysin or alveolysin. In some embodiments, the lytic agent can be a bacterial hemolysin such as listeriolysin O (LLO), a biologically active variant thereof, or a homolog thereof (e.g., a homolog found in a fungus or higher eukaryotic organism). More generally, where we suggest the inclusion of a specific agent, it is to be understood that this encompasses a teaching of any isoform or homolog of that agent (including lytic peptides or any other agent described herein as useful in the present moieties). LLO is a sulfhydryl-activated pore-forming toxin, which is a major virulence factor required for the escape of bacteria from phagosomal vacuoles and entry into the host cytosol. After binding to target membranes, LLO undergoes a major conformation change, leading to its insertion in the host membrane and formation of an oligomeric pore complex. LLO is synthesized as a 529 amino acid precursor. The 25 amino acid signal peptide is cleaved to generate the mature form. The N-terminal region is not required for secretion and hemolytic activity, but is involved in phagosomal escape of bacteria in infected cells and is critical for bacterial virulence. This region also contains a PEST-like sequence, which controls listeriolysin O production in the cytosol. An exemplary LLO can have the amino acid sequence found in GenBank at public GI number GI:46906434.

The bacterium Listeria monocytogenes produces the unique protein LLO as a tool for endosomal escape from phagosomes in macrophages. Thus, LLO and other proteins that similarly facilitate escape of the therapeutic agent are useful in the present compositions and methods. What makes LLO unique is that unlike other lysins it is only active within the lysosomal compartment. Once the bacterium and protein are released into the cytoplasm, LLO is inactivated through a variety of mechanisms (Schnupf et al., 2007 Microbes Infect. 9:1176-1187, 2007), the most important of which is due to pH sensitivity (Geoffroy et al., Infect. Immun. 55:1641-1646, 1987). LLO and other lysins have been used previously as tools for the delivery of macromolecules including DNA and proteins (Sun et al. J. Controlled Release 148:219-225, 2010; Giles et al., Nucleic Acids Res. 26:1567-1575, 1998, Walev et al., Proc. Natl. Acad. Sci. USA 98:3185-3190, 2001, Provoda et al., J. Biol. Chem. 278:35102-35108, 2003). In fact, LLO has even been used as the cytotoxic component of an immunotoxin (Bergelt et al., Protein Sci. 18:1210-1220, 2009). Our invention relates to our hypothesis that targeted versions of these gelonin and listeriolysin O would synergize through enhanced intracellular delivery.

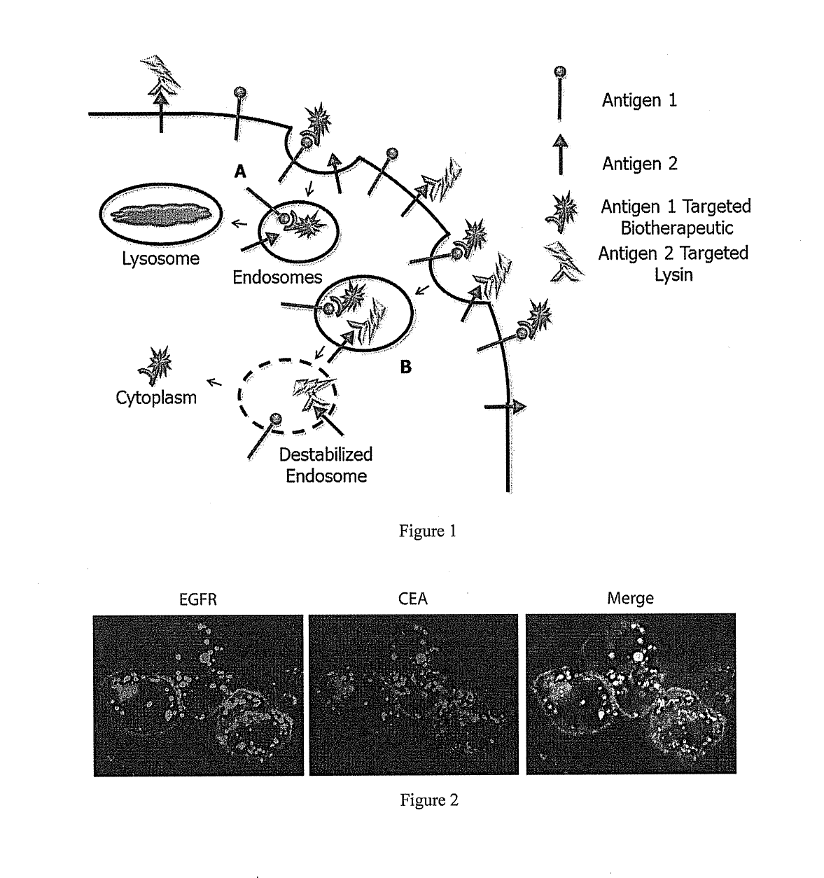

In the studies described below, we synthesized LLO as a fusion protein with the same Fn3 domains targeting the same set of antigens used for our immunotoxins. These fusion proteins were shown to have specific binding to antigen positive cells similar to that shown by their immunotoxin counterparts. When antigen positive cells were treated with both immunotoxin and targeted-LLO, significant potentiating effects were observed. This in trans approach to intracellular delivery is a departure from traditional intracellular delivery methods in which the membrane disruptive agent and the therapeutic payload are directly connected. FIG. 1 shows a schematic diagram depicting the way in which two different agents targeting the same cell, in this illustration through two different antigens, can become colocalized within the same endosomal compartment where the potentiating moiety facilitates the release of the therapeutic agent or “payload”.

The lytic agent can also be a eukaryotic polypeptide, for example, a perforin. Perforin is a cytolytic protein found in the granules of CD8 T-cells and NK cells. Upon degranulation, perforin inserts itself into the target cell's plasma membrane, forming a pore. Perforin is a synthesized as a 555 amino acid precusor protein. The 21 amino acid signal peptide is cleaved to generate the mature form. Perforin includes an MACPF domain, generally including amino acids 27-375; an EGF-like domain, generally including amino acids 376-408 and a C2 domain, generally including amino acids 416-498. The lytic membrane-inserting part of perforin is the MACPF domain, a region that has some homology with cholesterol-dependent cytolysins from Gram-positive bacteria. Perforin also has structural and functional similarities to complement component 9 (C9). Like C9, perforin creates transmembrane tubules and is capable of lysing a variety of target cells. Thus, C9 or biologically active variants thereof can also be incorporated as a lytic agent in the compositions described herein. Perforin is one of the main cytolytic proteins of cytolytic granules, and is a key effector molecule for T-cell- and natural killer-cell-mediated cytolysis. In one embodiment, a first portion of the potentiating moiety can be perforin or a biologically active variant thereof. A exemplary perforin can have the amino acid sequence found in GenBank at public GI number GI:40254808.

It is generally appreciated that the C-terminal domain of cytolysins is important for activity. Accordingly, where a biologically active fragment or other variant of a cytolysin is employed, the C-terminal domain can remain unmodified or largely unmodified (e.g., it may be at least 90% (e.g., at least 95, 97, 98, or 99%) identical to its wild type correlate.

The lytic agent can also be an endosome-disruptive peptide. Some endosome-disruptive peptides can integrate into the endosomal membrane in acidic environments and adopt a secondary or tertiary structure that generates an opening, for example a pore, in the endosomal membrane. Exemplary endosome-disruptive peptides include GALA, KALA and melittin.

GALA is a synthetic pore-forming peptide having a repeated peptide motif ‘EXLA’ which exists as a random coil in aqueous solutions above pH 5 and forms an amipathic α-helix in solution at pH 5 and below. Generally, suitable GALA peptides include at least 10 amino acids, typically 20-100 amino acids. An exemplary GALA peptide, also known as GALA 30, can have the amino acid sequence: WEAALAEALAEALAEHLAEALAEALEALAA (SEQ ID NO:19). GALA peptides solvated in aqueous solution at neutral pH, do not form α-helices because of the electrostatic repulsions between the glutamic acid residues. However, at pH 5, the neutralization of the glutamic acid residues promotes the formation of an amphipathic α-helix and GALA binding to lipid bilayers, such as endosome membrane. In the optimal pH range of 5 and below, GALA induces the leakage of the endosome membranes and rapid changeover in membrane structure (flip-flop of phospholipids). GALA peptides of various sizes may be synthesized by known methods, including those of Nicol et al., Biophys. J., 76:2121-2141, 1999.

KALA is a cationic peptide with a major repeat sequence of ‘KALA.’ KALA exists as a random coil in aqueous solutions above pH 5 and forms an amipathic α-helix in solution at pH 5 and below. An exemplary KALA peptide can have the amino acid sequence: WEAKLAKALAKALAKHLAKALAKALAKALKACEA (SEQ ID NO:20).

Other pore-forming peptide having demonstrated membrane-disrupting properties include JTS1, which can have the amino acid sequence, GLFEALLELLESLWELLLEA (SEQ ID NO:21), and melittin, which can have the amino acid sequence: GIGAVLKVLTTGLPALISWIKRKRQQ (SEQ ID NO:22).

The lytic agent can also be a molecule that is not a polypeptide. In some embodiments, the lytic agent can be a saponin. Saponins are plant glycosides composed of a steroid or triperpenoid aglycone core and one or more sugars that are covalently linked to the aglycone. Glucose, galactose, glucoronic acid, xylose and rhamnose are commonly bound monosaccharides. Saponins have membrane permeabilizing properties. These pore-forming properties depend generally on the amount of membrane-bound cholesterol. The saponin platycodin D directly interacts with cholesterol but not with triglycerides, suggesting that that the interactions of saponins might be specific for steroids. In addition to cholesterol, the sugar chains also affect pore formation. Pore formation is independent of membrane cholesterol in saponins with two sugar side chains, but cholesterol-dependent in those without sugars. Moreover, saponins possessing two sugar side chains generally are less membrane permeable than those with only one side chain. A saponin can be incorporated in the present compositions regardless of side chain number.

In some embodiments, the lytic agent can be an amine functionalized polymer, for example, polyethylene imine (PEI), polylysine, poly(amidoamine) dendrimers, poly(L-lactide-co-L-lysine), poly(serine ester), poly(4-hydroxy-L-proline ester), poly[α-(4-aminobutyl)-L-glycolic acid], poly(4-hydroxy-L-proline ester), poly[α-(4-aminobutyl)-L-glycolic acid], and poly(beta-amino esters).

Regardless of the particular structure, the lytic agent can be linked to the binding agent in any manner, including by way of the peptide bonds, covalent bonds, and non-covalent complex associations described herein. A linker can be any reagent, molecule or macromolecule that connects a first agent (e.g., a therapeutic agent) to a second agent (e.g., a lytic agent and/or binding agent) such that a) the linkage complex is stable under physiological conditions; and b) the connection between the agents do not alter the relevant biological abilities of the agents (e.g., the connection between a linker and a lytic agent does not substantially affect the capacity of the lytic agent to disrupt intracellular membranes).

Therapeutic Agents:

The therapeutic agent can be any agent that would, under ideal circumstances, be internalized and directed to the cytoplasm to confer efficacy. To assess the efficacy of the present system, the therapeutic agent can be applied to a cell in vitro, either alone or linked with a binding agent, and the amount of the therapeutic agent that reaches the cytoplasm can be compared with the amount that reaches the cytoplasm when the therapeutic agent is applied at varying concentrations in concert with a potentiating moiety and, optionally, a clustering agent (as noted herein, the present system can be configured such that the multi-specific binding agent(s) that induce clustering of their cell surface targets can be included within a clustering agent per se or included in the therapeutic and/or potentiating moieties to achieve the same end with fewer separate or individual components). The efficacy of the present system can be assessed relative to what one would observe in the absence of a potentiating moiety or in the absence of potentiating and clustering moieties.

For example, the therapeutic agent can be a toxin, including a naturally occurring or man-made toxin. In other instances, the therapeutic agent can be an antigen, a hormone, an enzyme, a growth factor (including an interleukin), or any other protein-based therapeutic. In some embodiments, the therapeutic agent can be an antibody. Because antibodies typically are secreted from cells and function in an extracellular environment, useful antibodies will generally be those that have been engineered to function intracellularly. Such “intrabodies” can include antibodies with any or all of the following modifications: single chain antibodies (scFvs), modification of immunoglobulin VL domains for hyperstability, antibodies resistant to the more reducing intracellular environment, or antibodies that are expressed as a fusion proteins with a stable intracellular proteins, e.g., maltose binding protein.

A toxin can be a protein-based toxin, for example, an enzymatically active toxin of bacterial, fungal, plant or animal origin or synthetic toxins, or fragments thereof. Exemplary bacterial toxins include diphtheria toxin, pseudomonas exotoxin, anthrax or botulinum toxin types A, B, C, D, E, F or G, cholera toxin, pertussis toxin, shiga toxin, bordetella pertussis AC toxin. Exemplary plant toxins include gelonin and ricin.

In some embodiments, the therapeutic agent can be a cytotoxic molecule, e.g., a chemotherapeutic agent. Administration of such agents using the compositions and methods of the invention may provide enhanced specificity and a targeted, dose-sparing effect, which is useful for anti-cancer agents with a narrow therapeutic index such as the anthracycline antibiotics (e.g., doxorubicin, epirubicin, and daunomycin).

Other useful therapeutics include those agents that promote DNA-damage, such as double stranded breaks in cellular DNA, in cancer cells. Any form of DNA-damaging agent known to one of ordinary skill in the art can be used. DNA damage can typically be produced by radiation therapy and/or chemotherapy. Examples of radiation therapy radioactive iodine (¹²⁵iodine or iodine¹³¹ strontium⁸⁹, or radioisotopes of phosphorous, palladium, cesium, iridium, phosphate, or cobalt. Examples of DNA-damaging chemotherapeutic agents include, without limitation, busulfan (Myleran™), carboplatin (Paraplatin™), carmustine (BCNU), chlorambucil (Leukeran™), cisplatin (Platinol™), cyclophosphamide (Cytoxan™, Neosar™), dacarbazine (DTIC-Dome™), ifosfamide (Ifex™), lomustine (CCNU), mechlorethamine (nitrogen mustard, Mustargen™), melphalan (Alkeran™), and procarbazine (Matulane™). Other cancer chemotherapeutic agents include, without limitation, alkylating agents, such as carboplatin and cisplatin; nitrogen mustard alkylating agents; nitrosourea alkylating agents, such as carmustine (BCNU); antimetabolites, such as methotrexate; folinic acid; purine analog antimetabolites, mercaptopurine; pyrimidine analog antimetabolites, such as fluorouracil (5-FU) and gemcitabine (Gemzar□); hormonal antineoplastics, such as goserelin, leuprolide, and tamoxifen; natural antineoplastics, such as aldesleukin, interleukin-2, docetaxel, etoposide (VP-16), interferon alfa, paclitaxel (Taxol□), and tretinoin (ATRA); antibiotic natural antineoplastics, such as bleomycin, dactinomycin, daunorubicin, doxorubicin, daunomycin and mitomycins including mitomycin C; and vinca alkaloid natural antineoplastics, such as vinblastine, vincristine, vindesine; hydroxyurea; aceglatone, adriamycin, ifosfamide, enocitabine, epitiostanol, aclarubicin, ancitabine, nimustine, procarbazine hydrochloride, carboquone, carboplatin, carmofur, chromomycin A3, antitumor polysaccharides, antitumor platelet factors, cyclophosphamide (Cytoxin□), Schizophyllan, cytarabine (cytosine arabinoside), dacarbazine, thioinosine, thiotepa, tegafur, dolastatins, dolastatin analogs such as auristatin, CPT-11 (irinotecan), mitozantrone, vinorelbine, teniposide, aminopterin, caminomycin, esperamicins (See, e.g., U.S. Pat. No. 4,675,187), neocarzinostatin, OK-432, bleomycin, furtulon, broxuridine, busulfan, honvan, peplomycin, bestatin (Ubenimex™), interferon-β, mepitiostane, mitobronitol, melphalan, laminin peptides, lentinan, coriolus versicolor extract, tegafur/uracil, and estramustine (estrogen/mechlorethamine).

Other useful therapeutic agents include produgs, which are precursors or derivative forms of a pharmaceutically active substance that is typically less cytotoxic or non-cytotoxic to tumor cells compared to the parent drug and is capable of being enzymatically activated or converted into an active or the more active parent form. Prodrugs amenable to delivery by the present compositions and methods include, but are not limited to, phosphate-containing prodrugs, thiophosphate-containing prodrugs, sulfate-containing prodrugs, peptide-containing prodrugs, D-amino acid-modified prodrugs, glycosylated prodrugs, b-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use herein include, but are not limited to, those chemotherapeutic agents described above.

Any polypeptide that interacts with and blocks the biological activity of a target required for cell viability can be used with the methods of the invention. The polypeptide can be an antibody that specifically binds to an intracellular target related to cell viability, for example, a polypeptide that mediates apoptosis such as p53 and the members of the p53 pathway. Alternatively, or in addition, the polypeptide can target protein kinases involved in oncogenic signalling, for example, the ErbB family. Useful antibodies in this case might be those that specifically bind to intracellular domains of ErbB family members, for example, Her2 or antibodies that specifically bind to cytoplasmic polypeptides in the downstream signalling pathways, PI3K-Akt and MAPK.

The MAPK pathway and its relationship with the Erbb family is well described. Its activation leads to the transcription of genes that drive cellular proliferation, as well as migration, differentiation and angiogenesis. Signalling through the PI3K-Akt pathway leads to several cellular end points, and survival and anti-apoptosis signalling are two main outcomes. There are three classes of PI3Ks, of which class 1A members are primarily responsible for mediating the signals generated by activation of growth factor receptors 1. Class 1A family members are heterodimers that consist of a p85 regulatory subunit, which is crucial for mediating activation through Erbb dimers, and a p110 catalytic subunit. The activation and phosphorylation of growth factor receptors facilitates the membrane recruitment of a signalling complex, which in this case consists of PI3K, which binds directly to the phosphotyrosine residues of the relevant Erbb moiety or to Erbb-bound adaptor proteins such as a GRB2-Sos (son of sevenless)-Ras complex (not shown) through the p85 subunit. Activation of the Akt family, which are serine-threonine kinases, allows the activation through phosphorylation of many other proteins that initiate processes to enable cell survival, suppression of apoptosis and cell cycle control. Exemplary polypeptides in the MAPK pathway include SHc, GRB2, Ras, Raf, MEK and MAPK. Exemplary polypeptides in the PI3K-Akt pathway include mTOR, p27, BAD, NF-kB and GSK3B.

Nucleic acid-based therapeutics can also be delivered by the compositions and methods described herein. These therapeutics include any RNA-based therapeutics, including antisense oligonucleotides, microRNAs, and any RNA-based therapeutics that mediate RNAi (e.g., an siRNA or shRNA). RNAi is a powerful technique used to downregulate or “knockdown” the expression of a target gene at the mRNA level in a cell. With current computational algorithms, and databases of validated siRNA designs (Krueger et al., Oligonucleotides 17:237-250, 2007), virtually an gene can be a target for RNAi. This makes it possible for RNAi by siRNA to address a wide range of possible disease targets, including cancer, autoimmune disease, and infectious diseases (Morrissey et al., Nature Biotechnol. 23:1002-1007, 2005; Okumura et al., Proc. Natl. Acad. Sci. USA 105:3974-3979, 2008; Ptasznik et al., Nature Med. 10:1187-1189, 2004; Kim et al., J. Controlled Release 129:107-116, 2008; Xia et al., Pharm. Res. 24:2309-2316, 2007; Song et al., Nature Med. 9:347-351, 2003).

The primary challenge preventing siRNA-based therapeutics from experiencing wide-spread use in the clinic is a problem of delivery to the cell cytoplasm where it is biologically active. Additionally, in order to improve the potency of a given siRNA payload, and to prevent off-target effects, the cell type of interest is preferably specifically targeted via cell-specific surface receptors or other surface molecules. The technology described herein addresses both issues while maintaining other properties that may provide advantages over current technologies.

The systems described herein, whether described with reference to a nucleic acid-based therapeutic agent or any other type of therapeutic agent, were developed to make the cell receptor target and the target (e.g., the gene target for RNAi) modular. This allows targeting of any gene and surface molecule for which siRNAs and targeting agents can be generated, respectively. As an example, we have chosen three model receptor targets of high interest in cancer therapy: CD25, CEA, and EGFR. CD25 is expressed at high levels in T cell lyphomas, as well as in regulatory T cells, which can inhibit anti-tumor immune responses (Sakaguchi et al., J. Immunol. 155:1151-1164, 1995; Jones et al., Clin. Cancer Res. 10:5587-5594, 2004; Curiel, J. Clin. Invest. 117:1167-1174, 2007). CEA, which is expressed in cancers of the gastrointestinal tract, and EGFR, which is expressed in a wide variety of cancers, were both recently prioritized in the top 15 target cancer antigens for translational research (Cheever et al., Clin. Cancer Res. 15:5323-5337, 2009).

As noted, the compositions and methods described herein can be used to deliver a therapeutic agent that mediates RNA interference. The large majority of vehicles for targeted delivery of short interfering RNA (siRNA) employ the use of nanoparticle-based delivery vehicles (Jarvis, Chem. Eng. News 87:18-27, 2009), with small molecule or macromolecular ligands tethered to the nanoparticle surface for targeting and internalization. Although nanoparticle-based delivery vehicles can carry large siRNA payloads, they suffer from several problems that limit their efficacy, and that protein-based delivery can potentially solve. They are rapidly phagocytosed by the reticuloendothelial system and accumulate in the liver and spleen, leading to poor pharmacokinetics and biodistribution (Alexis et al., Mol. Pharm. 5:505-515, 2008). They also exhibit poor extravasation and penetration into solid tumors due to their large size (Schmidt and Wittrup, Mol. Cancer. Ther. 8:2861-2871, 2009). On the other hand, protein-based systems can be engineered to fall within the window of 60 kDa and 500 kDa, which would be large enough to avoid rapid renal clearance, yet small enough for efficient extravasation and avoiding phagocytic clearance. Moieties having a molecular weight of about 60 kDa to about 500 kDa (e.g., about 75, 100, 150, 200, 250, 300, 350, 400, and 450 kDa) are within the scope of the present invention. Also, nanoparticle formulations are difficult to prepare in a reproducible and monodisperse manner. In contrast, proteins are relatively straightforward to synthesize using recombinant DNA technology, and can generally be purified in a straightforward, reproducible manner to monodispersity. Accordingly, the present compositions and methods feature a multi-agent (e.g., two, three, or four-agent) approach in which all of the agents employed can be proteinaceous and which targets agents that mediate RNAi (and other therapeutics) to target cells (e.g., those expressing a cancer cell antigen) using a non-polycationic carrier. As described elsewhere herein, in some instances, the functionality carried by two (or more) of the moieties described herein can be collapsed into a singly moiety.

While limited in number, there are protein-based delivery vehicles for siRNA that combine a targeting agent, such as an antibody fragment, with an siRNA complexation agent, usually a short polycationic peptide (Kumar et al., Cell 134:577-586, 2008; Song et al., Nat. Biotechnol. 23:709-717, 2005; Peer et al., Proc. Natl. Acad. Sci. USA 104:4095-4100, 2007; and Winkler et al., Mol. Cancer. Ther. 8:2674-2683, 2009). However, these methods have limitations that have prevented them from reaching the potential of protein-based delivery methods. For example, they suffer from poor pharmacokinetics and biodistribution, due to high global organ uptake as a result of their high positive charge (Niesner et al., Bioconjug. Chem. 13:729-736, 2002; Lee and Pardridge, Bioconjug. Chem. 12:995-999, 2001). They also tend to be inefficient and require large amounts of siRNA for delivery (Kumar et al., supra; Peer et al., supra). These agents require complex preparation or purification schemes, such as protein refolding (Winkler et al., supra), or chemical conjugation (Kumar et al., supra). Also, in our experience, these polycationic peptides are prone to aggregation and are generally poorly behaved and difficult to work with.

As is true for any other therapeutic agent delivered by the present methods, a two-agent or three-agent delivery system can be employed for RNA-based therapeutics. As is true for any other therapeutic agent that exerts its effect after successful delivery to the cytoplasm of a cell, RNA-based therapeutics can be delivered with or without being a part of a larger moiety that includes a binding agent. Thus, the invention encompasses compositions (e.g., pharmaceutical compositions) that include an RNA-based therapeutic, which may or may not be joined to a binding agent, and a potentiating moiety; methods of treating a cell, tissue, or patient by administering a therapeutically effective amount of such a composition to the cell, tissue, or patient; and methods of treating a cell, tissue, or patient by administering the RNA-based therapeutic and the potentiating agent separately (e.g., at different times and/or by different routes of administration). Where a third agent is employed, that agent can be a clustering agent that induces clustering of a cell surface molecular target. While the clustering moiety is described further below, we note here that it can be multi-specific (e.g., bi- or tri-specific), and one or more of the binding agents within the clustering moiety can be the same as (or can bind the same epitope as) the binding agent included in the potentiating moiety. While the moieties work together, each makes a distinct contribution to more effective drug delivery.

In delivering an RNA-based therapeutic (which may mediate RNAi), the therapeutic moiety can be a targeted fusion protein (or other assembled moiety, such as a protein conjugate or non-covalent complex) that includes a double stranded RNA binding domain (dsRBD) as a non-polycationic siRNA carrier. The dsRBD moiety binds reversibly and specifically to double stranded RNA, and provides protection against siRNA degradation by RNases (Kim et al., J. Gene. Med. 11:804-812, 2009). Fusion proteins comprising dsRBD can deliver siRNA to the endosomes of a target cell (in Example 3, we work with an EGFR-expressing cell line).

Thus, therapeutic moieties designed to deliver an RNA-based therapeutic can include a binding agent (as described elsewhere herein), a carrier to which the RNA-based therapeutic is bound, and the therapeutic itself. Like other therapeutic moieties, an RNA-based therapeutic moiety can also include an accessory sequence, such as a linker or an Fc region of an immunoglobulin. The carrier can be non-polycationic; the carrier can reversibly and specifically bind double- or single-stranded RNA. For example, the carrier can include an RNA recognition motif. The carrier may be, or may be derived from, a naturally occurring RNA-binding protein that regulates the translation of RNA or that normally binds RNA in the context of a post-transcriptional event (such as RNA splicing). For example, in addition to dsRBD and biologically active fragments or other variants thereof, an RNA carrier can be, or can be derived from, a translation initiation factor, an snRNP, ADAR, and like proteins.

A potentiating moiety can contain an endosome-destabilizing protein (e.g., the cholesterol dependent cytolysin, perfringolysin O (PFO)) in order to enhance the endosomal escape of siRNA or any other RNA-based therapeutic. As demonstrated in Example 3, targeted delivery of PFO induced gene silencing in the A431 cell line in a dose-dependent and EGFR-dependent manner. Our studies with a three-agent system have indicated that the cytotoxicity of the cytolysin creates a therapeutic window which can be significantly expanded using a third agent that induces EGFR clustering, which in turn increases gene silencing potency and decreases the toxicity of PFO. Altogether, this system is potent, with only ˜10 nM siRNA required for gene silencing, with a wide therapeutic window, spanning approximately two orders of magnitude of targeted cytolysin concentrations.

Molecular Targets:

The binding agent within the present moieties can specifically bind any cell surface molecule, including a cell adhesion molecule or a cell surface receptor, and any of the moieties can be multispecific. For example, any given moiety can include a binding agent that specifically binds more than one distinct epitope on a molecular target (i.e., the binding agent can be bi- or tri-specific). Similarly any given moiety can include a plurality of distinct binding agents that bind distinct epitopes; e.g., a moiety can include a first modified fibronectin domain (or any other non-immunoglobulin or immunoglobulin binding agent) that binds a first epitope on a molecular target and a second modified fibronectin domain (or any other non-immunoglobulin or immunoglobulin binding agent) that binds a second epitope on a molecular target. The molecular targets may be distinct from one another. Immunoglobulin-based binding agents can be similarly employed, and the moieties can include both non-immunoglobulin and immunoglobulin-based binding agents. Preferably, these binding agents bind cell surface molecules that are selectively expressed on the surfaces of cells targeted for treatment (e.g., cells that are proliferating at an undesirable rate). For example, a binding agent can specifically bind a cell adhesion molecule such as CEA or growth factor receptors such as EGFR or IGFR, which serves as tumor markers. More specifically, the binding agent can specifically bind a polypeptide encoded by one of the following human genes or a non-human homolog of one of these genes: CEACAM1, CEACAM3, CEACAM4, CEACAM5, CEACAM6, CEACAM7, CEACAM8, CEACAM16, CEACAM18, CEACAM19, CEACAM20, CEACAM21, IGF1, and IGF2.

In other embodiments, the binding agent can specifically bind a cell surface receptor, including any tyrosine kinase receptor that is expressed or misexpressed in connection with cancer or another condition one wishes to treat with the present fusion proteins (e.g., an autoimmune disease). Useful tyrosine kinase receptors include members of the ErbB family: epidermal growth factor receptor (EGFR; also known as HER1), ERBB2 (HER2), ERBB3 (HER3) and ERBB4 (HER4). More generally, the molecular target can be a tyrosine kinase receptor. In addition to those in the ErbB family, the binding agents in the present moieties can include a receptor in the insulin, PDGF, FGF, VEGF, HGF, Trk, Eph, AXL, LTK, TIE, ROR, DDR, RET, KLG, RYK, or MuSK receptor family.

Binding agents that specifically bind to other cell surface antigens can also be used. The choice of antigen will depend, in part, on the particular disease that is being treated. In the case of cancer, particularly useful antigens are those that antigens whose expression is relatively restricted to tumor cells, for example, tumor-associated antigens (TAAs). Exemplary TAAs include RAGE, MART (melanoma antigen), MAGE (melanoma antigen) 1-4, 6 and 12, MUC (mucin) (e.g., MUC-1, MUC-2, etc.), tyrosinase, Ras, Pmel 17 (100), GnT-V intron V sequence (N-acetylglucoaminyltransferase V intron V sequence), prostate cancer psm, PRAME (melanoma antigen), β-catenin, MUM-1-B (melanoma ubiquitous mutated gene product), GAGE (melanoma antigen) 1, BAGE (melanoma antigen) 2-10, gp75 and lung resistance protein (LRP). As noted, the binding can specifically bind to a cell-surface polypeptide, but the invention is not so limited; binding agents that specifically bind to carbohydrates or lipids can also be used as binding agents in the moieties described herein.

We may describe the epitope to which a binding agent binds as an “alternative” epitope when it is exposed only in some circumstances (e.g., only in the case of a mutant or activated receptor). For example, a domain can specifically bind a cysteine loop at the end of the EGFR extracellular domain II, including a conformational epitope that is exposed only when the receptor transitions into the open conformation upon dimerization. Thus, binding agents that bind alternative epitopes can be incorporated into the moieties described herein.

Configurations:

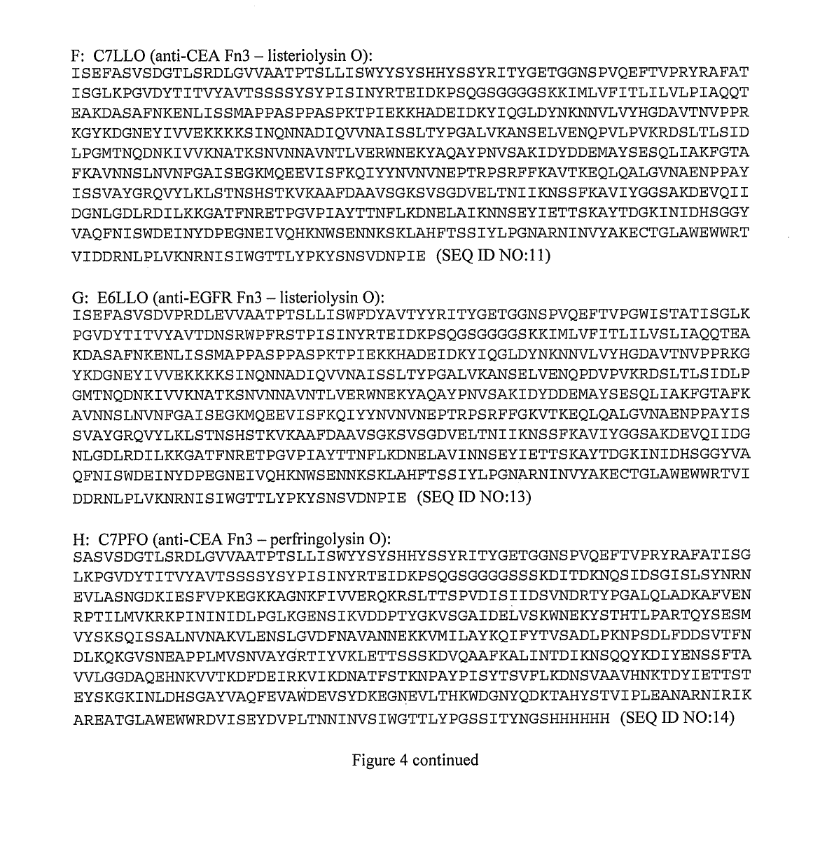

The agents within the moieties described herein (e.g., within a potentiating, therapeutic, or clustering moiety) can be variously configured and may also be joined in a variety of ways. For example, the agents within these moieties can be fused or otherwise linked. For example, the agents can be chemically conjugated, bound through affinity binding partners, or joined within a non-covalent complex. For example, a lytic agent or a therapeutic agent can be fused or otherwise linked (e.g., chemically conjugated) to a second amino acid sequence (a heterologous sequence) that binds to a molecule on the surface of the targeted cell (i.e., a binding agent). The agents within a fusion protein can be included in either order and/or either orientation. For example, where a fusion protein includes a lytic agent and a binding agent, the lytic agent can be “first” (i.e., at the amino-terminal end of the fusion protein) and the binding agent can be “second” (i.e., at the carboxy-terminal end of the fusion protein), or vice versa. The same is true where a fusion protein includes a therapeutic agent and a binding agent; the therapeutic agent can be “first (i.e., at the amino-terminal end of the fusion protein) and the binding agent can be “second” (i.e., at the carboxy-terminal end of the fusion protein). In addition, in either case, the first and second agents can be fused head-to-head, tail-to-tail, or head-to-tail. In all cases, there may be a linker (e.g., a polypeptide linker) between the first and second agents, as illustrated in one of more of the sequences shown in FIG. 4. However, constructs in which the first and second agents are fused directly to one another (i.e., that do not include a linker) are also within the scope of the invention.

The linker can vary in length. For example, it can vary from about 3 to about 30 amino acid residues. For example, it may include at least or about 3, 5, 10, 15, 20, or 25 amino acid residues. The residues may also vary and may include those having relatively small side chains, such as glycine and serine. The residues may be polar or non-polar, and linkers can include both polar and non-polar residues. The residues may also be naturally occurring, non-naturally occurring (e.g., selenocyteine), or a mixture of naturally- and non-naturally occurring residues.

As noted elsewhere herein, methods of making fusion proteins are well known in the art and can be carried out using standard recombinant techniques.

In other embodiments, the moieties described herein can be chemical conjugates. For example, a peptide-protein conjugate can be generated by the common one- or two-step conjugation methods known in the art; the one-step conjugation involving modification of a residue in a first agent (e.g., a proteinaceous binding agent) via an activated second agent, and the two-step conjugation involving the introduction of complementary chemoselective handles into both agents. In the one-step approach, one would simply introduce an activated synthetic handle onto one agent (e.g., a lytic peptide) and conjugate the peptide to a second agent (e.g., an immunoglobulin as a binding agent). This usually results in a distribution dependent on the interaction between particular residues. For example, where the activated synthetic handle is an active ester on a “first” agent, such as a peptide therapeutic or lytic peptide, interaction with a lysine residue on the second agent (e.g., a binding agent) can produce an amide link. Where the activated synthetic handle is an aldehyde or ketone, an imine link may be produced; where the activated synthetic handle is an alkyl halide, maleimide, or vinylsulfone, one can produce a thioester link with cysteine residues on the second agent. A transglutaminase can link glutamine with lysine through their sidechains. Other such conjugations will be known and understood by one of ordinary skill in the art. As noted, in a two-step conjugation, both agents are modified or pre-programmed such that specific conjugates result, with the distribution of one agent on another determined by the point(s) of addition of the “handle.”

Where the binding agent includes an immunoglobulin or a biologically active variant thereof, the binding agent can be joined to a peptide therapeutic or a lytic peptide according to the methods used to generate CovX-bodies (peptide-antibody conjugates). The complexity derived from the presence of the same sidechain functionalities in two components (e.g., an immunoglobulin-like binding agent and a peptide therapeutic) has driven the development of specific conjugation strategies, and newer conjugation methods are producing better defined and more easily manufactured conjugates. The strategies include the use of molecular biology techniques that render amino acid residues in one agent (e.g., a binding agent) more reactive toward a complementary reactive group that has been introduced into another agent (e.g., a peptide therapeutic or a lytic peptide) by chemical synthesis (as in the covX-body, for example).

In other embodiments, the two-agent system can be configured differently. For example, the binding agent incorporated in each moiety can be the same agent. That is, the same binding agent can be linked to the lytic agent (the binding agent and lytic agent together forming a potentiating moiety) and to a therapeutic (the binding agent and the therapeutic agent together forming a therapeutic moiety). In another aspect, the two-agent system can be consolidated into a single agent with a fusion protein or protein conjugate including all three of: a binding agent that specifically binds a cell surface molecule; a potentiating agent that destabilizes an intracellular compartment into which the agent has been directed; and a therapeutic agent. While the system can be alternatively configured as described here, our studies to date indicate that specificity and potency are better when a first moiety contains the potentiating agent and a second, distinct moiety contains the therapeutic agent.

The polypeptides of the invention can include a label, marker, or tag to facilitate detection or protein purification. Where the two binding agents include complimentary affinity tags, those tags can be incorporated into an agent as described herein (i.e., into a binding, lytic, therapeutic, or clustering agent) and used to bring the agents together to form a more complex moiety. Such labels, markers, and tags include those known in the art, including a “strep-tag.” The strep-tag is a synthetic peptide consisting of eight amino acids (Trp-Ser-His-Pro-Gln-Phe-Glu-Lys (SEQ ID NO:18). This peptide sequence exhibits intrinsic affinity towards Strep-Tactin, a specifically engineered streptavidin and can be N- or C-terminally fused to recombinant proteins. By exploiting the highly specific interaction, Strep-tagged proteins can be isolated in one step from crude cell lysates. Because the Strep-tag elutes under gentle, physiological conditions it is especially suited for generation of functional proteins.

If necessary to improve the solubility of any of the moieties described herein, they may include a polypeptide, such as a maltose binding protein. Such accessory sequences may be especially useful in moieties including cytolysins that are prone to aggregate.

With respect to the configuration of a clustering moiety, the various binding agents included (e.g., a tetrameric immunoglobulin, any other immunoglobulin-like molecule, including scFvs, Fab′ fragments, and F(ab′)2 fragments as well as non-immunoglobulin binding agents such as modified fibronectin domains) can be variously arranged. For example an scFv, Fab′, F(ab′)2, diabody, triabody, or modified fibronectin domain can be fused, directly or indirectly (e.g., via a linker), to one or both of the heavy chains of a tetrameric immunoglobulin. For example, an scFv, Fab′, F(ab′)2, diabody, triabody, or any combination of such immunoglobulin-like moieties can be fused to the amino termini and/or the carboxy termini of the heavy chain(s) of the tetrameric immunoglobulin. Alternatively, or in addition, the scFv, Fab′, F(ab′)2, diabody, triabody, or modified fibronectin domain can be fused to the amino termini and/or the carboxy termini of the light chain(s) of the tetrameric immunoglobulin. For example, in one embodiment, the binding moiety comprises a tetrameric immunoglobulin and a plurality of (e.g., four) scFvs or modified fibronectin domains. As shown in FIG. 6 (right-hand illustration), two of the plurality of binding agents can be linked to the amino termini of the heavy chains of the tetrameric immunoglobulin and two of the plurality of binding agents can be linked to the carboxy termini of the light chains of the tetrameric immunoglobulin. Thus, a clustering moiety can include a tetrameric immunoglobulin with scFvs fused to the amino termini of the heavy chains and scFvs (binding the same or a distinct epitope) fused to the carboxy termini of the light chains. One, two, three, or four of these scFvs can be, instead, any other suitable binding agent, including an Fab′ F(ab′)2, a diabody or triabody or any of the non-immunoglobulin binding domains known in the art, including the modified fibronectin domains illustrated herein. In other embodiments, the binding moiety can include a plurality of just one type of an immunoglobuin-based binding moieties. For example, the antibody-based constructs can include two, three, four, or more tetrameric immunoglobulins fused to one another (with the provisio that the antibody-based construct is not a naturally occurring immunoglobulin, such as an immunoglobulin of the M class). Similarly, two, three, four or more scFvs, Fab′ fragments, F(ab′)2 fragments, diabodies or triabodies can be joined to one another (e.g., via linkers).

To further illustrate how the moieties of the invention can be combined in pharmaceutical formulations (or concentrated formulations, such as solutions for dilution or lyophilized powders) and how they may be used in combination to carry out the methods of the invention, we have generated the Table below. In the first instance, the composition or method employs Agents A and B; in the second, the composition or method employs Agents A, B, and C; and so forth. In the ninth-12^{th} instances, we indicate tht more than one of each type of moiety can be employed. For example, two distinct therapeutic moieities and/or two distinct potentiating moieties can be employed. These instances serve to illustrate that point and other combinations of distinct moieties can also be included or employed and are within the scope of the present invention.

| | Agent A | Agent B | Agent C | Agent D | Agent E | Agent F | Agent G | Agent H |
|---|---|---|---|---|---|---|---|---|
| 1 | x | x | | | | | | |
| 2 | x | x | x | | | | | |
| 3 | | x | | x | | | | |
| 4 | | x | x | x | | | | |
| 5 | | | x | | x | | | |
| 6 | | x | | | | x | | |
| 7 | x | | | | | | x | |
| 8 | | | | x | | | x | |
| 9 | xx | x | | | | | | |
| 10 | xx | xx | | | | | | |
| 11 | x | xx | | | | | | |
| 12 | xx | x | x | | | | | |
| 13 | xx | xx | x | | | | | |
| 14 | x | xx | x | | | | | |
| 15 | | x | x | | | | | |
| 16 | | x | | | | | | |
| 17 | | | | | | | | x |

Nucleic Acids:

Nucleic acid (e.g., DNA) sequences coding for any of the polypeptides described herein, including potentiating fusion proteins and therapeutic fusion proteins, are also within the scope of the present invention, as are methods of making such nucleic acid constructs and the fusion proteins they encode. For example, a given gene sequence can be obtained (e.g., from a library, a depository, or a commercial source) and incorporated into an expression vector. One or both of the termini of any given gene sequence can be modified using known techniques that allow the sequence to be placed in an expression vector as desired. For example, a sequence encoding a polypeptide that selectively binds a cell surface moiety (e.g., a cell surface receptor) can be fused either upstream or downstream from a sequence encoding a polypeptide that selectively lyses intracellular membranes. Similarly, a sequence encoding a polypeptide that selectively binds a cell surface moiety (e.g., a cell surface receptor) can be fused either upstream or downstream from a sequence encoding a therapeutic protein. When necessary or desired, known mutagenesis techniques, such as those carried out by PCR methods, can be used to alter a DNA sequence. For example, mutagenesis techniques can be used for codon optimization, and any of the methods of making a nucleic acid construct or expressing a polypeptide of the invention can include the steps of providing a DNA sequence and/or mutagenizing a DNA sequence. Known techniques can also be used to alter a DNA sequence such that it differs from a naturally occurring sequence. As noted, polypeptides incorporated in the fusion proteins of the invention can be wild type polypeptides or biologically active fragments or other biologically active variants thereof. Thus, the invention encompasses nucleic acid sequences that have been modified to encode a non-naturally-occurring polypeptide that retains sufficient biological activity to be useful in the methods described herein. For example, a biologically active fragment or other biologically active variant of a cytolysin will retain the ability to lyse intracellular membranes to a useful extent (e.g., to such an extent that therapeutic macromolecules within such membranes can be released to the cytoplasm). In one embodiment, a wild type polypeptide (e.g., a wild type binding, lytic, or therapeutic moiety) can be subjected to random mutagenesis, and sequences encoding variants with the desired specificity or biological activity can be selected (e.g., from a phage library or by an in vitro assay for the activity desired (e.g., selective binding, lysis, or therapeutic effect).

The methods of generating nucleic acid constructs and/or expressing or constructing fusion proteins can be carried out using standard techniques known in the art. For example, one can use standard methods of protein expression (e.g., expression in cell culture with recombinant vectors) followed by purification from the expression system. Thus, the invention encompasses methods of generating a nucleic acid construct that includes a sequence encoding a fusion protein as described herein. The methods can be carried out by providing a nucleic acid sequence (e.g., a DNA sequence) that encodes the fusion protein and delivering it (e.g., in the context of an expression vector) to a host cell that is maintained under conditions that allow for the expression of protein from the nucleic acid construct. For example, the cell can be cultured at a temperature that permits cell survival and proliferation (e.g., between about room temperature and physiological temperature (i.e., about 37° C.)) and with a standard culture medium that supports cell survival and proliferation. Methods of culturing biological cells, including mammalian, bacterial, fungal, and insect cells, all of which can be employed in the present methods for the expression of a fusion protein, are well known in the art. In some circumstances (e.g., to produce shorter polypeptides, such as a given domain, linker, or tag), chemical synthesis can also be used. Recombinant and synthetic methods can be used alone or in combination to produce the fusion proteins described herein, whether the component parts of those fusions are naturally occurring or biologically active fragments or other variants thereof. While the constructs are not limited by the manner in which they achieve a desired outcome, we expect fragments and other variants of a given binding moiety to retain the ability of the wild type correlate to specifically bind a specified molecular target, such as a tyrosine kinase receptor, although the affinity for the target may vary. Similarly, we expect fragments and other variants of a given lytic moiety to lyse intracellular (e.g., endosomal) membranes, although their lytic capacity may vary relative to the wild type correlate. Therapeutic proteins can also vary from a wild type correlate as long as they have or retain the ability to achieve a desired therapeutic outcome.

More specifically, to produce a fusion protein (e.g., an immunoglobulin such as an scFv linked to a lytic moiety or a therapeutic moiety) or a portion thereof (either of which may optionally include an accessory sequence, linker, tag, or any other optional component), nucleic acid sequences encoding the desired polypeptide can be ligated into an expression vector and used to transform a prokaryotic cell (e.g., bacteria) or transfect a eukaryotic (e.g., insect, yeast, plant, or mammalian) host cell. In general, nucleic acid constructs can include a regulatory sequence operably linked to a nucleic acid encoding a fusion protein, and the nucleic acid constructs of the invention encompass those with regulatory sequences including promoters, enhancers, polyadenylation signals, and/or terminators. These elements can be included as needed or desired to affect the expression of a nucleic acid sequence. The transformed or transfected cells can then be used, for example, for large or small scale production of a given fusion protein (or a component part thereof) by methods well known in the art. In essence, such methods involve culturing the cells under conditions suitable for production of the polypeptide encoded by the nucleic acid used and isolating the polypeptide from the cells or from the culture medium. To facilitate purification, the expressed fusion protein can be biotinylated.

Pharmaceutical Formulations, Methods of Treatment, and Conditions Amenable to Treatment:

The system described herein and the compositions within it are useful in delivering therapeutic agents to the cytoplasm of a cell. In some instances, employing the present system may improve delivery relative to other methods, including those carried out in the absence of a potentiating agent or binding agent(s). Therefore, the present methods include methods for improving delivery or improving existing or other yet-to-be-determined treatment methods. In the methods of the invention, the various moieties (e.g., a therapeutic moiety and a potentiating moiety) may be delivered either simultaneously or sequentially by the same or different routes of administration. For example, the potentiating moiety can be delivered first by intravenous infusion and the therapeutic moiety can be delivered afterward by intravenous infusion or another route of administration circumventing first pass metabolism (e.g. retro-orbital injection or intraperitoneal injection).

Upon reaching a target cell, both moieties (i.e., a potentiating fusion protein and a therapeutic fusion protein) must be internalized. However, our studies suggest that internalization need not be simultaneous. For example, a therapeutic fusion protein can be administered first and come to reside in a sub-cellular compartment from which it would be released upon the subsequent administration of a potentiating fusion protein. For example, following internalization, a therapeutic colocalized with a potentiator in a sub-cellular compartment will be released into the cytoplasm as the lytic moiety (e.g., LLO) is activated in response to reduced pH.

The moieties described herein can be administered directly to a mammal. Generally, the moieties can be suspended in a pharmaceutically acceptable carrier (e.g., physiological saline or a buffered saline solution) to facilitate their delivery. Encapsulation in a suitable delivery vehicle (e.g., polymeric microparticles or implantable devices) may increase the efficiency of delivery. Compositions (e.g., pharmaceutical and/or physiologically acceptable compositions) can be made by combining any of the moieties provided herein with a pharmaceutically acceptable carrier. Such carriers can include, without limitation, sterile aqueous or non-aqueous solutions, suspensions, and emulsions. Examples of non-aqueous solvents include mineral oil, propylene glycol, polyethylene glycol, vegetable oils, and injectable organic esters. Aqueous carriers include, without limitation, water, alcohol, saline, and buffered solutions. Preservatives, flavorings, and other additives such as, for example, antimicrobials, anti-oxidants (e.g., propyl gallate), chelating agents, inert gases, and the like may also be present. It will be appreciated that any material described herein that is to be administered to a mammal can contain one or more pharmaceutically acceptable carriers, and those just specifically mentioned can be used in any combination (e.g., a moiety can be formulated in a non-aqueous suspension with a preservative, and an antimicrobial agent). In particular embodiments, the moieties of the invention are formulated in the same manner as a commercially developed binding agent (such as an antibody, including cetuximab and others described above) or therapeutic (such as a chemotherapeutic agent or RNA-based therapeutic).

The pharmaceutical formulations described herein can be administered to any part of the host's body for subsequent delivery to a target cell. A composition can be delivered to, without limitation, the brain, the cerebrospinal fluid, joints, nasal mucosa, blood, lungs, intestines, muscle tissues, skin, or the peritoneal cavity of a mammal. Thus, the invention encompasses pharmaceutical compositions suitably formulated for delivery to the brain, the cerebrospinal fluid, joints, nasal mucosa, blood, lungs, intestines, muscle tissue, skin, or the peritoneal cavity. In terms of routes of delivery, a composition can be administered by intravenous, intracranial, intraperitoneal, intramuscular, subcutaneous, intramuscular, intrarectal, intravaginal, intrathecal, intratracheal, intradermal, or transdermal injection, by oral or nasal administration, or by gradual perfusion over time. In a further example, an aerosol preparation of a composition can be given to a host by inhalation.

As is understood in the art, the dosage required will depend on the route of administration, the nature of the formulation (including the manner in which various agents are combined into moieties), the nature of the patient's illness, the patient's size, weight, surface area, age, and sex, other drugs being administered, and the judgment of the attending clinician. Suitable dosages are expected to be in the range of 0.01-1,000 μg/kg. Wide variations in the needed dosage are to be expected in view of the variety of cellular targets and the differing efficiencies of various routes of administration. Variations in these dosage levels can be adjusted using standard empirical routines for optimization, as is well understood in the art. Encapsulation of the moieties in a suitable delivery vehicle (e.g., polymeric microparticles or implantable devices) may increase the efficiency of delivery.

As is known in the art, dosage and treatment regimens may also vary based on the condition to be treated. One of ordinary skill in the art wishing to use the present system can obtain information and guidance regarding dosage from currently available therapeutics, including antibody therapeutics. For example, cetuxamab, when used for the treatment of colorectal cancer in adults is delivered intravenously at 400 mg/m² as an initial loading dose, which is administered as a 120-minute infusion (with a maximum rate of infusion of 10 mg/minute). The recommended weekly maintenance dose is 250 mg/m² infused over 60 minutes (with a maximum rate of infusion of 10 mg/minute). For treatment of squamous cell carcinoma of the head and neck, in adults, the recommended delivery for cetuxamab is intravenous in combination with radiation therapy. The recommended dose is 400 mg/m² as a loading dose, which is given as a 120-minute infusion (with a maximum infusion rate of 10 mg/min) one week prior to initiation of a course of radiation therapy. The recommended weekly maintenance dose is 250 mg/m² infused over 60 minutes (again, with a maximum infusion rate of 10 mg/min) weekly for the duration of radiation therapy (typically 6 to 7 weeks). Ideally, administration should be complete one hour prior to radiation therapy. As a single agent, the recommended initial dose is 400 mg/m² followed by 250 mg/m² weekly (with the maximum infusion rate being 10 mg/minute). While the moieties described herein may be administered at dosages comparable to those of known cancer biotherapeutics, such as cetuximab, the dosages may also be lower. Accordingly, pharmaceutical formulations having, and methods of treatment using, doses less than those recommended for cetuximab are within the scope of the present invention.

The patient treated may have, or the medicament prepared may be useful in treating, breast cancer, bladder cancer, lung cancer, including non-small-cell lung cancer, colorectal cancer, squamous-cell carcinoma of the head and neck, ovarian cancer, cervical cancer, esophageal cancer, salivary gland cancer, gastric cancer, a B cell cancer, multiple myeloma, thyroid cancer, a glioblastoma, or pancreatic cancer.

The duration of treatment with any composition provided herein can be any length of time from as short as one day to as long as the life span of the host (e.g., many years). For example, a moiety or moieties of the invention can be administered once a week (for, for example, 4 weeks to many months or years); once a month (for, for example, three to twelve months or for many years); or once a year for a period of five years, ten years, or longer. It is also noted that the frequency of treatment can be variable. For example, the system can be employed once (or twice, three times, etc.) daily, weekly, monthly, or yearly.

An effective amount of any composition provided herein can be administered to an individual in need of treatment, and the methods of the invention encompass methods of treatment. The steps of the method can include identifying a patient in need of treatment and administering, to the patient, an effective amount of a composition described herein. It should be apparent that the precise composition and the active engineered protein therein will be selected based on the type of illness afflicting the patient; the protein must target a molecular entity (e.g., a cell surface receptor) that is mis-expressed in connection with the illness. The term “effective” as used herein refers to any amount that induces a desired response while not inducing significant toxicity in the patient. Such an amount can be determined by assessing a patient's response after administration of some known amount of a particular composition. In addition, the level of toxicity, if any, can be determined by assessing a patient's clinical symptoms before and after administering some known amount of a particular composition. It is noted that the effective amount of a particular composition administered to a patient can be adjusted according to a desired outcome as well as the patient's response and level of toxicity. Significant toxicity can vary for each particular patient and depends on multiple factors including, without limitation, the patient's disease state, age, tolerance to side effects, and the availability of alternative treatments.

In any of the methods of treatment, the subject can be a human and the method can include a step of identifying a patient for treatment (e.g., by performing a diagnostic assay for a cancer or other condition described herein). Further, one may obtain a biological sample from a patient and expose cancerous cells within the sample to one or more moieties ex vivo to determine whether or to what extent the proteins internalize a target expressed by the cells or inhibit their proliferation or capacity for metastasis. Thus, the diagnostic and therapeutic methods of the invention can be described as methods for assessing the expression of a target in a cell; methods for inhibiting unwanted cellular proliferation, and/or methods for inhibiting metastasis. Similarly, one may obtain a biological sample from a patient and expose cancerous cells within the sample to one or more of the proteins that have been engineered to carry toxic cargo. Evaluating cell survival or other parameters (e.g., cellular proliferation or migration) can yield information that reflects how well a patient's cancer may respond to in vivo treatment with the moiety or moieties tested in culture. The patient identified as a candidate for treatment with the present engineered proteins may be one who is resistant to treatment with a conventional tetrameric immunoglobulin (e.g., cetuximab).

Any method known in the art can be used to determine if a particular response is induced. Clinical methods that can assess the degree of a particular disease state can be used to determine if a response is induced. The particular methods used to evaluate a response will depend upon the nature of the patient's disorder, the patient's age, and sex, other drugs being administered, and the judgment of the attending clinician.

Kits:

The compositions described herein can also be assembled in kits, together with instructions for use. For example, the kits can include measured amounts of a potentiating moiety comprising (a) a binding agent that specifically binds a cell surface molecule, (b) a lytic agent that destabilizes the membrane of an intracellular compartment and (c) optionally, a linker between the binding agent and the lytic agent. The kit can also comprise a therapeutic agent or a therapeutic moiety comprising a binding agent and a therapeutic agent. The potentiating moiety and the therapeutic agent or therapeutic moiety can be packaged together in the same container. Alternatively, the potentiating moiety and the therapeutic agent or therapeutic moiety can be packaged separately for administration at different times or to permit more precise dose titration of each moiety. The potentiating moiety can be supplied as a sterile liquid or as a lyophilized suspension. Where one or more of the packaged moieties are fusion proteins (e.g., where a binding agent and the lytic agent are expressed as a fusion protein and constitute the potentiating moiety), the kit can include lyophilized host cells that have been engineered to express the fusion protein. Similarly, the therapeutic agent or therapeutic moiety can be supplied as a sterile liquid or as a lyophilized suspension. Where the therapeutic moiety comprises a polypeptide, the kit can include lyophilized host cells that have been engineered to express that polypeptide.

The kits can also include measured amounts of a clustering moiety. The clustering moiety can be packaged separately or in combination with either the potentiating moiety or the therapeutic agent or therapeutic moiety. Where the potentiating moiety and the therapeutic agent or therapeutic moiety are packaged together, the clustering moiety can also be packaged in concert. As is the case for the potentiating moiety and the therapeutic moiety, the clustering moiety can be supplied as a liquid or a lyophilized solution. And, where the clustering moiety is a fusion protein, the kit can include lyophilized host cell that express that fusion protein.

The packaging materials can include any or all of the following: packaging materials, for example, vials, packets, containers, syringes, buffers, culture media and antibiotics, devices and reagents for protein purification, for example, chromatography columns, filters, control proteins for quality assurance, and physiologically acceptable carriers. The kits can also include control reagents for treating or monitoring the condition for which prophylaxis or treatment is required.

The instructions for use can be conveyed by any suitable media. For example, they can be printed on a paper insert in one or more languages or supplied audibly or visually (e.g., on a compact disc) or provided as internet-based instructions accessible on the world wide web.

### EXAMPLES

### Example 1

### In Vitro Analysis of Selected Potentiating and Therapeutic Moieties; Independently Targeted Cytolysin and Gelonin Immunotoxins Combine to Exhibit Mechanistic Synergy

Below, or elsewhere herein, we may use the following abbreviations. “IT” for immunotoxin; “rGel” for recombinant gelonin; “Fn3” for the engineered 10^{th} Type III human fibronectin domain; “LLO” for listeriolysin O; “PFO” for perfringolysin O; “CDC” for cholesterol-dependent cytolysin; “EGFR” for epidermal growth factor receptor; “CEA” for carcinoembryonic antigen; “CI” for combination index; “SAF” for synergy assessment factor; and “TN₅₀” for the number of molecules required to incur a 50% loss of viability.

Useful Reagents and Methods:

Cell Lines.

In order to work with cells that variously express antigens that can be targeted by the agents of the invention, we selected four different established human cancer cell lines. HT-1080 is a human fibrosarcoma cell line negative for CEA. HT-1080(CEA) is a transfected variant of HT-1080 which expresses CEA at high levels (˜2×10⁶ copies/cell) on its surface through expression of the pIRES-CEA plasmid, which is maintained under selective pressure from geneticin. Both HT-1080 and HT-1080(CEA) also express ˜1×10⁵ copies of EGFR. A-431 is a human epidermoid carcinoma cell line that expresses high levels of EGFR (˜3×10⁶ copies/cell) but not CEA. HT-29 is a human colorectal carcinoma cell line that expresses lower levels of both CEA and EGFR (˜4×10⁵ copies/cell). As noted above, the fusion proteins of the invention, and the binding agents in particular, specifically bind molecules expressed on the cell surface, and these include cancer antigens or markers, such as CEA, and tyrosine kinase receptors, such as EGFR.

Construction of Expression Plasmids.

A gene encoding the lytic proteins LLO and PFO were codon-optimized for expression in E. coli and ordered from GenScript (Piscataway, N.J.). The genes were amplified by PCR using primers designed with 3′ complimentarity to either end. After being purified from an agarose gel, the gene products were inserted into the same fusion construct used previously for our immunotoxin synthesis by the method of Geiser et al. (Geiser et al., Biotechniques 31:88-90, 2001). The resulting plasmid encoded an open reading frame including, from N-terminus to C-terminus, maltose binding protein, N₁₀ linker, a Factor Xa protease site, an engineered 10^{th} Type III fibronectin domain, a G₄S linker, and LLO or PFO. Additionally, our PFO gene was truncated to remove an unnecessary secretory sequence and its construct modified to incorporated a tobacco-etch virus protease site N-terminal to the constant Factor Xa site.

Protein Expression and Purification.

Immunotoxins were synthesized as described previously (Pirie et al., J. Biol. Chem. 286:4165-4172, 2011), and Fn3-LLO/PFO production was conducted in a similar manner. Briefly, the appropriate plasmids were transformed into Rosetta (DE3) E. coli (Novagen, San Diego, Calif.) and grown on LB agar plates supplemented with ampicillin and chloramphenicol. Colonies were isolated from the plate and used to inoculate 15 mL of selective media which was then incubated overnight at 37° C. Cultures were then used to inoculate 1 L of antibiotic free LB media and allowed to grow to an OD₆₀₀˜0.5, at which point 5 mL of 0.1M IPTG was added and the cultures moved to incubate at 30° C. for 6 hours. Following induction, cultures were centrifuged at 15,000×g for 12 minutes, the supernatant removed, and the cell pellets frozen at −20° C. Pellets were resuspended in 25 mL amylose column buffer containing Complete EDTA-free protease inhibitor (Roche, Indianapolis, Ind.) and then sonicated on a Branson Sonifier 450A (Branson Ultrasonics, Danbury, Conn.) at 50% duty cycle and power level 6 for three, one-minute intervals. The resulting solution was centrifuged at 50,000×g for 30 minutes to pellet cell debris and the supernatant was applied to an amylose column as described by the manufacturer (New England Biolabs, Ipswich, Mass.). Purified recombinant proteins were concentrated and buffer exchanged into Factor Xa digestion buffer using Amicon columns with a 100 kDa MWCO then incubated overnight at 4° C. with 5 μl Factor Xa (New England Biolabs). Fn3-cytolysin was isolated from the cleaved MBP and Factor Xa by ion-exchange chromatography with a HiTrap Q column (GE Healthcare, Piscataway, N.J.). Larger scale protein production required for in vivo experiments was accomplished using the same protocol executed at 2 L and 10 L scales in Bioflo bioreactors (New Brunswick Scientific, Edison, N.J.) with oxygen control supplied by filtered air at 0.5 (vvm) and agitation adjusted to maintain dissolved oxygen levels above 30%. pH was controlled at 7.0 using 6N NaOH. Cells were grown to OD600=0.8 before induction as before. Purification was carried out as described previously.

Antigen Binding Affinity Titration.

Fn3-LLO fusions were biotinylated using amine reactive EZ-Link Sulfo-NHS-LC-biotin (Pierce, Rockford, Ill.). Antigen positive cell lines HT-1080(CEA) and A431 were lifted from culture plates with trypsin and resuspended in 4% formalin for 30 minutes before being washed and stored in phosphate buffered saline with 1% (w/v) bovine serum albumin (PBSA). Fixed cells were incubated with varying concentrations of biotinylated fusion protein overnight at 37° C., washed once and resuspended in 250 μL of PBSA with 1 μL streptavidin-phycoerythrin conjugate (Sigma, St. Louis, Mo.) for 1 hour at 4° C. Cells were washed once again and resuspended in 150 μL PBSA before being analyzed for fluorescence on an Accuri C6 flow cytometer (Accuri Cytometers, Ann Arbor, Mich.). For each titrating concentration, the median fluorescent intensity was determined and data sets for each immunotoxin were fitted to a standard binding isotherm using least-squares regression.

To titrate Fn3-PFO fusions, CEA (R&D Systems, Minneapolis, Minn.) was biotinylated using amine reactive EZ-Link Sulfo-NHS-LC-biotin (Pierce). Biotinylated CEA was loaded streptavidin coated magnetic beads (Invitrogen, Carlsbad, Calif.), and incubated with varying concentrations of C7 PFO for 6 hours at 4° C. EGFR 404SG ectodomain was expressed on the surface of yeast (Kim et al., Proteins 62:1026-1035, 2006), and incubated with varying concentrations of E6 PFO for 6 hours at 4° C. Cells or beads were washed once and resuspended in 50 μL of PBSA with 0.25 μL of rabbit anti-His6 antibody (Abcam, Cambridge, Mass.) which was labeled with the Alexa 647 Microscale Protein Labeling Kit (Invitrogen) for 30 minutes at 4° C. Cells or beads were washed twice with 200 μL PBSA before being analyzed for fluorescence by flow cytometry.

Hemolysis.

We conducted experiments to determine the degree to which Fn3-LLO (which we may also refer to as “Fn3-cytolysin) fusions would disrupt red blood cell membranes using a method similar to that described by Henry et al. (Biomacromolecules 7:2407-2414, 2006). Briefly, purified mouse red blood cells (Fitzgerald, Acton, Mass.) were washed twice with PBSA at either pH 7.4 or pH 5. Cells were mixed with Fn3-LLO fusions suspended in the same buffer to a final cell concentration of ˜1×10⁹ cells/ml and varying fusion concentration. Cells and protein were incubated at 37° C. for 1 hour before treatments were centrifuged at 13,500×g for 5 minutes. Supernatants were transferred to 96-well plates and absorbance read at 540 nm. Results were normalized to a PBS negative control and 1% Triton-X100 positive control.

Colocalization Microscopy.

1×10⁵ HT-29 cells were cultured on MatTek dishes with 0.13 mm coverslip bottoms in 200 μl of growth media to which Alexa584-sm3E anti-CEA antibody (7 μM, DOL 1.85) and Alexa488-225 anti-EGFR antibody (5 uM, DOL 6) were added with final concentrations of 11.7 nM and 33 nM, respectively. The cells were maintained in this solution under standard culture conditions (37° C., 5% CO₂) for 15 hours, after which the cells were washed three times with PBS and returned to growth media for 30 minutes before imaging. Cells were serum starved prior to treatment.

Cytotoxicity.

Log-phase tumor cells were removed by trypsinization, counted, and seeded on 96-well plates at 2,500 cells per well. Cells were allowed to adhere overnight, after which fresh growth media containing varying concentrations of Fn3-LLO fusion and/or immunotoxin was added to triplicate wells. Cells were incubated in treatment media for 48 hours before being replaced with media containing the WST I reagent (Roche). The red/ox solution was allowed to develop for 1-3 hours under normal culture conditions after which plates were measured for absorbance at 450 nm. Untreated cells and cells lysed with a 1% Triton-X100 solution were used as positive and negative controls, respectively. Measurements were set to baseline on negative control and normalized to positive control treatments, triplicates averaged, and standard deviations calculated. Delayed dose or time dependent cytotoxicity data were obtained by treating cells as described, removing treatment containing media, washing once with PBS, then replacing with fresh media or media containing the second agent for wells at each time point then following identical assay procedures after 48 hours. In situations where one agent was titrated and the other was fixed or where both agents' concentrations were fixed, the fixed concentration was selected so as to be non-toxic in the absence of the other agent.

Quantitative internalization. Cell lines were incubated with immunotoxins directly labeled with AlexaFluor 488 and unlabeled Fn3-LLO fusions. At various times, cells were washed with PBS and incubated for 30 minutes with the quenching rabbit anti-AlexaFluor 488 (Invitrogen, Carlsbad, Calif.). Cells were then scraped from the wells, washed again with PBS, and analyzed for internal fluorescent signal. Quantum Simply Cellular anti-Mouse IgG beads (Bangs Laboratories, Fishers, Ind.) with different quantified binding capacities were incubated with AlexaFluor 488 labeled mouse IgG for 30 minutes, washed with PBS, then measured for fluorescence. The number of fluorescent molecules per protein on both immunotoxins and mouse IgG was determined using absorbance measurements at 280 and 494 nm. Bead fluorescence measurements were used to generate a standard curve for fluorescence signal per fluorescent molecule. Immunotoxin internalization data were quantified by mapping fluorescence signal to the bead fit converting signal to fluorescent molecules and then translated into immunotoxin molecules using the labeling ratio.

Internalized Cytotoxicity.

Data obtained in time dependent cytotoxicity experiments were combined with those from quantitative internalization experiments and plotted to suggest the former as the dependent variable and the latter as the independent variable. Accumulated results were fitted using non-linear least squares regression of an exposure-response curve with variable slopes using MATLAB (The MathWorks, Natick, Mass.). From this fitting, a mid-range response metric was calculated and reported.

Statistical Analyses of In Vitro Data.

To quantify the extent of synergy between gelonin immunotoxins and targeted cytolysins in vitro we used designed cytotoxicity co-titrations to calculate combination index and cumulative data to calculate synergy assessment factor. The combination index metric was first used to determine the synergistic effects of mutually exclusive and mutually non-exclusive enzyme inhibitors by Chou and Talalay (Chou and Talalay, Trends in Pharmacological Sciences 4:450-454, 1983; and Chou and Talalay, Adv. Enzyme Regul. 22:27-55, 1984).

Synergy assessment factor is a more recent treatment of synergistic effects which was inspired by combination index. It was first put forth by Yan et al. as it pertained to synergy within signaling networks (Yan et al., BMC Syst. Biol. 4:50, 2010) and is equivalent to the fractional product equation described by Webb (Webb, Enzyme and Metabolic Inhibitors 55-79 (1963) at <New York: Academic Press>).

For CI calculations we simultaneously titrated immunotoxins and used 0.9 fraction affected as the analysis point.

$$\frac{[ IT ]}{{IT}_{90}} + \frac{[ P ]}{{P}_{90}} + \frac{[ IT ] \times [ P ]}{{( IT , P )}_{90}}$$

Alternatively, when using SAF we chose to calculate the metric for all titrations and data points then averaged for cell line or immunotoxin/potentiator of interest.

F_{A}(IT,P)−F_{A}(IT)×F_{A}(P)

Fn3-LLO fusion characterization. Fusion proteins of the Fn3-LLO type were designed with targeting to EGFR (E6LLO) and CEA (C7LLO). The fusions were expressed in E. coli at 0.5 mg/L. The Fn3 clones from which the fusions were derived were E626 and C743, which bind to EGFR and CEA with K_{d}'s of 260 pM and 1.8 nM, respectively. Fn3 clones were engineered for affinity to EGFR and CEA as described previously (Hackel et al., J. Mol. Biol. 381:1238-1252, 2008). We determined the affinity titration of the two fusions. Non-linear regression fitting shows that the K_{d}'s for the fusion proteins are 5.01 nM for E6-LLO (which targets EGFR) and 4.09 nM for C7-LLO (which targets CEA). More specifically, biotinylated Fn3-LLO fusions were titrated against fixed A431 or HT-1080(CEA) cells. Cells were washed and incubated for 1 hour on ice with streptavidin-phycoerythrin and then assayed by flow cytometry. Fusions showed binding affinity K_{d}'s in the low nM range, only slightly reduced relative to the parent Fn3 affinity.

Given our understanding of LLO/cytolysin activity and the work of Bergelt et al. (Protein Sci. 18:1210-1220, 2009), we realized that it was important to assess the direct cytotoxicity of the Fn3-LLO fusions. The independent cytotoxicity of the fusions was tested by titration on a variety of antigen positive and antigen negative cell lines. Fitting the data with concentration-response curves with variable slopes gave IC₅₀ values that correlated inversely with antigen expression level (see the Table below). These data show that targeted LLO and PFO fusions do indeed possess some inherent cytotoxicity and that, much like gelonin fusions, its cytotoxicity is related to its ability to specifically bind cell surface antigens.

| IC₅₀'s (M) | Cell Line | | |
|---|---|---|---|
| Potentiator | HT-1080(CEA) | A431 | HT-29 |
| C7LLO | 8.3 × 10⁻⁸ | — | >1 × 10⁻⁶ |
| E6LLO | 2.5 × 10⁻⁷ | 7.1 × 10⁻⁸ | >1 × 10⁻⁶ |
| C7PFO | 7 × 10⁻¹² | 9.8 × 10⁻¹² | 1.2 × 10⁻¹⁰ |
| E6PFO | 1 × 10⁻¹¹ | 5.4 × 10⁻¹⁰ | 1.5 × 10⁻¹¹ |

A common assay in the characterization of bacterial CDCs and other membrane disruptive materials is the hemolysis assay. The ability of Fn3-cytolysin fusions to disrupt red blood cells at either physiological or endosomal pH can be representative of non-specific toxicity and activity respectively. We were particularly interested in assessing hemolysis because of work suggesting very low toxicity limits of LLO in vivo Geoffroy et al.; Infect Immun 55:1641-1646, 1987. At pH 7, the EC₅₀ for membrane disruption by E6LLO was ˜500 nM, while at pH 5 it was ˜3 nM. For E6 PFO the EC₅₀'s were 25 pM and 4 pM respectively. This data is consistent with work by Jones and Portnoy that queried LLO and PFO properties and found similar hemolytic characteristics (Jones and Portnoy, Infect. Immun. 62:5608-5613, 1994).

Results:

Colocalization of Antigen Targets.

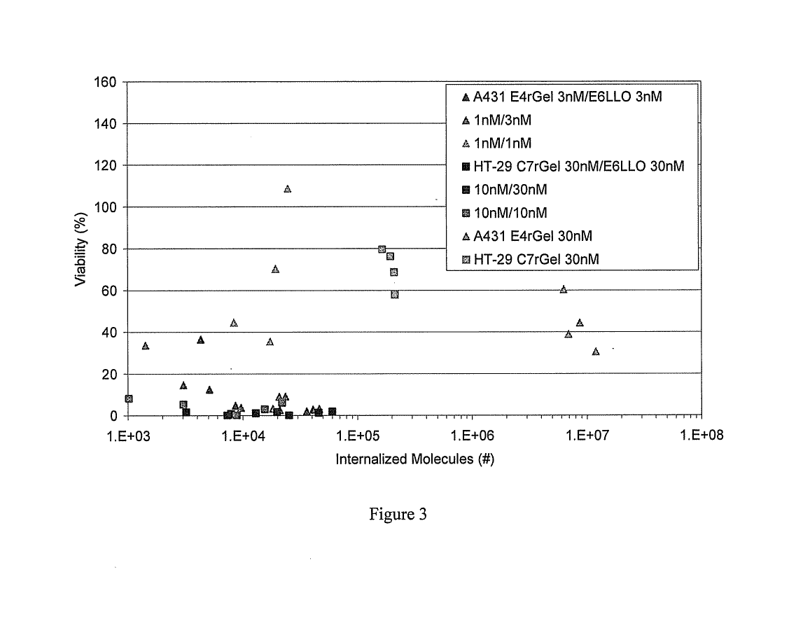

HT-29 cells express approximately 1×10⁵ copies of both EGFR and CEA on their surface. These cells were treated with fluorescently labeled anti-EGFR IgG and anti-CEA scFv. Subsequent microscopy images showed that both antigens were bound and internalized showing punctuate staining (FIG. 2). Further, the merging of the images from the two fluorescent channels indicates strong colocalization. Colocalization was characterized using image analysis software and found to have a positive Pearson's coefficient of correlation.

Potentiated Immunotoxin Cytotoxicity In Vitro:

In this study, we used two different immunotoxins, E4rGel and C7rGel targeting EGFR and CEA, respectively, that have been described previously (Pirie et al., J. Biol. Chem. 286:4165-4172, 2011). These immunotoxins show independent IC₅₀'s of approximately 30 nM and 5 nM respectively on cell lines expressing high levels of antigen. However, on HT-29 cells that express low levels of antigen, the immunotoxins appear no more potent than untargeted toxin with IC₅₀'s around 1 μM. Remarkably, when we titrate the immunotoxins in the presence of non-toxic levels of Fn3-LLO/cytolysin, the potency is increased by several orders of magnitude. Remarkably, when we titrate the immunotoxins in the presence of non-toxic levels of Fn3-cytolysin, the potency is increased by several orders of magnitude. We find that on high antigen expressing cells, the IC₅₀ of E4rGel is decreased from 30 nM to ˜50 pM and that of C7rGel is potentiated from about 1 nM to the single digit pM range. Perhaps most importantly, we were able to show that otherwise ineffective immunotoxin can be activated to an equivalent degree on HT-29 cells where the IC₅₀s shift from 1 μM to 1 nM. This shift was consistent for both non-competitively co-targeted immunotoxin and potentiator and for differentially targeted components.

To statistically support the observed synergy between gelonin and cytolysin immunotoxins we employed two different metrics: combination index (CI) and synergy assessment factor (SAF). Qualitatively, CI values are characterized as antagonistic when >1, additive when =1, and synergistic when <1. Alternatively, SAF will =0 when a combination is additive, >0 when antagonistic, and <0 when synergistic. We report here examples of CI₉₀ values less than 1 and negative SAF values indicative of the strong synergy we observed across all cytolysin fusions when combined with gelonin immunotoxins on any cell line. The strength of each synergy metric tended to show an inverse correlation with the independent potency of the gelonin immunotoxin on the cell line in question.

Delayed Exposure Cytotoxicity.

Much like differential targeting of two antigens, we envisioned a system in which the two agents might be dosed independently in vivo to prevent non-specific uptake of both agents simultaneously by antigen-negative cells. We tested the possibility of this approach in vitro by treating cells for a fixed period of time with growth media containing one agent, then removing it and replacing it with new media containing the appropriate second agent. A431 cells were treated with anti-EGFR immunotoxin for 12 hours and then non-competitive anti-EGFR potentiator for 24 hours with the potentiator dose being delayed 0, 12, 24, or 48 hours. The order of treatment was also switched, with potentiator exposures of 12 hrs followed by immunotoxin exposures of 12 hrs delayed by the same periods. In FIG. 6B similar treatments are made on HT-29 cells albeit with differentially targeted immunotoxin (CEA) and potentiator (EGFR) at order of magnitude higher concentrations. Treatment concentrations for each agent were non-toxic when exposed independently. As we might expect, loss of viability is maximized for treatments made simultaneously and decreases incrementally as the separation between treatments is increased. For low concentration (3 nM and/or 10 nM) treatments on high-antigen expressing cells, we found that potentiation is almost completely absence when the two treatments are separated by 48 hours. However, on the low-antigen expressing cells, higher concentration (30 nM and/or 100 nM) treatments with differentially targeted agents resulted in significant potentiation and loss of viability even when treatments were separated by 48 hours.

Internalized Cytotoxicity with Reduction of TN₅₀.

Previously, we have shown that there is an intracellular barrier to immunotoxin potency which requires that ˜5×10⁶ toxin molecules are internalized before a cell will undergo apoptosis (Pirie et al., J. Biol. Chem. 286:4165-4172, 2011). Furthermore, it was established that this barrier was common to all gelonin immunotoxins tested regardless of cell type, antigen targeted, or binding affinity. Here, we utilized the same techniques to characterize the intracellular barrier in the presence of a potentiating moiety. We observed the expected increase in the number of anti-CEA immunotoxins internalized by HT-29 cells with respect to treatment concentration and incubation time. The number of immunotoxins internalized is significantly less than what would be necessary to cause cytotoxicity in the absence of potentiator, and yet we observed loss of viability in treatment matched cells. However, the number of immunotoxins internalized is still significantly less than would be necessary to induce cytotoxicity in the absence of potentiator, and yet loss of viability is observed in treatment matched cells

When we combine the data from internalization and cytotoxicity measurements, we get internalized cytotoxicity curves that show TN₅₀ values less than 10⁴ molecules, indicating a several order of magnitude drop in the barrier due to the presence of potentiator. In fact, we are unable to directly ascertain the true TN₅₀ in the presence of potentiator because the fluorescent signal from so few molecules is indiscernible from autofluorescence of the cells. To give a sense of the magnitude of the enhancement of intracellular delivery due to potentiator we plotted internalized cytotoxicity measurements for anti-CEA immunotoxins on HT-1080(CEA) and HT-29 cells in the presence of CEA or EGFR potentiator respectively alongside the curve-fit for unpotentiated TN₅₀ from our previous work (Pirie et al.).

### Example 2

### In Vivo Analysis

Model System.

All in vivo studies used 6-8 week old athymic N_{cr} (nu/nu) nude mice obtained from Taconic (Hudson, N.Y.). This is the standard model for National Cancer Institute studies and many pharmaceutical and oncology screening programs. Their outbred background originates from BALB/c and NIH(s) stocks.

Dose Escalation.

Dose escalation was carried out for all in vivo applied fusion proteins using the canonical “3+3 method” in which three mice are dosed at a particular concentration and if no dose limiting toxicity is observed then the dose is raised, if one of three mice exhibit limiting toxicity then a new cohort of three mice are treated at the same dose, and if two or more mice show limiting toxicity then escalation is stopped and the previous dose is deemed the maximum tolerated dose. Here, in the absence of any informed basis on which to select a starting dose, a logarithmically spaced escalation was employed where after limiting toxicity was observed a linear escalation was continued from the last tolerated dose.

Plasma Clearance.

Fusion proteins were labeled with Li-Cor 800CW dye (Li-Cor Biosciences, Lincoln, Nebr.) by N-hydroxyl succinimide ester reaction with free lysines. Labeled proteins were injected at their respective maximum tolerated doses into three mice and blood samples taken at logarithmically spaced time points by tail clipping. Blood was collected into heparin coated capillary tubes and imaged on a Li-Cor Odyssey Imaging System (LiCor Biosciences). Imaging sensitivity was adjusted so as to maximize signal-to-noise ratio without saturating the fluorescence channel. Fluorescent signals were averaged across mice at each time point and fitted with a bi-exponential function for retro-orbital injections and with a tri-exponential for intraperitoneal injections to determining absorption and clearance constants as well as plasma half-lives.

Dose Separation.

Using clearance information as a basis we applied the same “3+3 method” as for dose escalation to determine the minimum separation time between doses. Gelonin immunotoxin was dosed first by retro-orbital injection at its independent maximum tolerated dose. After the defined amount of time targeted cytolysin was dosed at its own independent maximum tolerated dose either by retro-orbital injection in the opposite eye or by intraperitoneal injection. Mice were monitored for toxicity for 72 hours following the second injection.

Tumor Xenograft Growth Inhibition.

Mice received subcutaneous injections of 3×10⁶ HT-29 cells in the right flank on day 0. Digital caliper measurements to determine tumor volume were begun on day 5 and taken every other day for the duration of the study. On day 7 measurements were used to divide mice to treatment groups so as to balance the average tumor volume for each group. Four groups were used: PBS/PBS, C7rGel/PBS, PBS/E6 PFO, and C7rGel/E6 PFO as primary/secondary respectively. Treatments in the first study shown were given on day 7 and 11 with 3 hour and 6 hour separations between primary retro-orbital injection and secondary intraperitoneal injection. In the second study doses were separated by 6 hours in both rounds.

Results:

Independent In Vivo Dosing and Clearance of Gelonin or Cytolysin Immunotoxins:

Nude mice were dosed by the “3+3 method” with increasing amounts of either gelonin or cytolysin immunotoxins until dose limiting toxicity was observed. For CEA-targeted C7rGel, no substantial toxicity was observed up to doses of 16 mg/kg while for E6LLO and E6 PFO dose limiting toxicities were reached above 0.6 and 0.2 mg/kg respectively. Maximum tolerated doses were used for all subsequent experiments.

We evaluated plasma half-lives of our therapeutic proteins to better inform dose separation. Bi-exponential fitting of protein clearance data from retro-orbital injections yielded alpha-phase half-lives and beta-phase half-lives of 30 minutes and 12.2 hours for C7rGel, 124 minutes and 11.5 hours for E6LLO, and 34 minutes and 13.3 hours for E6 PFO. Tri-exponential fitting of data from intraperitoneal injections revealed a plasma absorption half-time of 65 minutes.

Combination Treatment of Murine Xenografts:

Dual agent treatment was performed initially with retro-orbital injection of both agents with C7rGel injected first and E6LLO or E6 PFO injected second. We shrank separation time linearly, again using the “3+3 method” until dose separation limiting toxicity was observed. Results indicated that when using this route of administration, E6LLO could only be dosed no sooner than 12 hours after C7rGel injection and the minimum delay with E6 PFO was 24 hours. Based on these limitations and our understanding of the clearance rate of C7rGel, we investigated the possibility of intraperitoneal injection of targeted cytolysins secondary to injection of gelonin immunotoxin. We hoped that like injection of other protein therapeutics into the peritoneal cavity we might be able to achieve a more gradual uptake into the plasma while gelonin immunotoxins were still residing in the tumor (Barrett et al., Cancer Res. 61:3434-3444, 1991). When administering C7rGel by retro-orbital injection followed by intraperitoneal injection of targeted cytolysin we found that E6LLO and E6PFO doses could follow as little as 6 hours and 3 hours after, respectively.

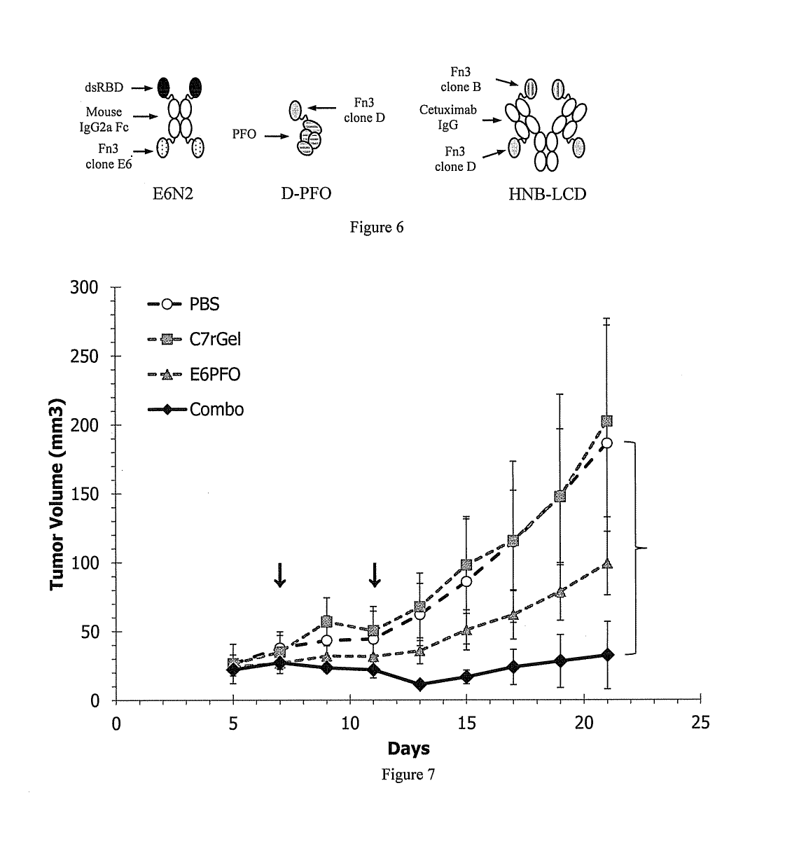

Once we had determined the maximum tolerated dose and minimum dose separation time we used these to test efficacy in a tumor xenograft model. When mice were given both therapeutic agents in succession, they were found to localize in the tumor interstitium and their combined presence in the tumor resulted in apoptosis not observed in PBS treated tumors. To investigate tumor growth inhibition, nude mice bearing developed HT-29 colorectal carcinoma xenografts were dosed with either phosphate buffered saline (PBS), C7rGel & PBS, PBS & E6 PFO, or C7rGel & E6 PFO at days 7 and 9 post tumor injection. While individual therapeutic treatments were unable to control tumor growth, the combination treatment resulted in statistically significant inhibition of tumor progression (FIG. 7).

Discussion:

The importance of targeted intracellular delivery to the advancement of numerous therapeutic agents is well appreciated (Varkouhi et al., J. Controlled Release 151:220-228, 2011). While there has been some use of cholesterol-dependent cytolysins in this area, our studies are the first to use LLO as a targeted in trans delivery agent together with a therapeutic that targets a second, different cell-surface target. One benefit of this approach is that targeting two independent antigens may improve in vivo tissue specificity. Additionally, when two agents are delivered in trans, they can be dosed independently, which can reduce unwanted side effects.

Our two agent approach was conceived of with the intention of directing a therapeutic macromolecule and a potentiator towards different antigen targets. While it may be more convenient to target both components (i.e., the therapeutic agent, such as Fn3-rGel, and a potentiating agent, such as Fn3-LLO or Fn3-PFO) to the same cell-surface molecule, and while such a design would ensure colocalization, we were interested in taking advantage of the additional specificity that targeting two different antigens might confer in vivo. Prior to doing so we sought to confirm that our two antigens of interest would colocalize within endosomes. We found that in HT-29 cells, CEA and EGFR will colocalize. Moreover, scaling analysis reveals that, in the absence of a preferential internalization mechanism, two antigens are more likely to colocalize than not. Examining the current system where an average sized cell expresses ˜10⁵ copies of either antigen per cell, if we assume a random surface distribution of antigens and unbiased internalization, then each endosome would contain on average 40 copies of each antigen.

We have described the synthesis of novel targeted fusion proteins incorporating Fn3 and LLO or PFO. These fusions are expressed in E. coli and can be readily purified. Fn3 clones affinity matured for binding EGFR and CEA retained most of their affinity in the potentiator constructs, conserving nM binding constants. In cytotoxicity tests these potentiators showed antigen specificity and moderate cytotoxicity with IC₅₀'s around 100 nM. Analysis of the hemolytic activity of the fusions showed consistency with previous reports of the different cytolysins' activity. LLO fusions showed significant pH dependence and optimum activity at endosomal pH while PFO fusions exhibited greater potency but limited pH dependence. The EC₅₀'s at physiological pH corresponded roughly with the non-specific cytotoxicity, while that at endosomal pH was consistent with the antigen positive cell potentiating activity.

We proceeded to validate the ability of our Fn3-cytolysin to mediate increased cytoplasmic delivery. When our immunotoxins targeting EGFR and CEA were titrated in the presence of Fn3-cytolysin targeting the same antigen competitively or non-competitively, or targeting an independent antigen, the immunotoxins displayed IC₅₀'s several orders of magnitude lower than in the absence of the potentiating agent. Potency at these scales significantly improves the forecast for use of type I immunotoxins in the treatment of cancer. The degree to which potency of a particular targeted therapeutic macromolecule is enhanced by Fn3-cytolysin will be directly tied to its inherent potency in the cytoplasm. We believe that these enhancements in potency and selectivity will be broadly applicable to the delivery of any targeted therapeutic macromolecule without its own translocation mechanism.

Having established a metric for determining the relative barrier to cytoplasmic delivery (Pirie et al. J. Biol. Chem. 286:4165-4172, 2011), we were interested in determining how Fn3-cytolysin might lower this barrier. Fundamentally, our assay queries the integrity of subcellular compartments in general, because it does not consider specific subcellular localization and merely approximates cytoplasmic release with cytotoxicity. Thus, an agent capable of enhancing the release of immunotoxin through either pore formation or membrane destabilization should quantitatively differentiate itself in this assay. We find that Fn3-LLO fusions are so effective in this setting as to reach the lower limit of detection for the method. In fact, so few molecules were required to be internalized prior to observing cytotoxicity that the signal from the internalized immunotoxins did not surpass background fluorescence of the cells. As a result we are forced to approximate the TN₅₀ for our immunotoxins in the presence of potentiating Fn3-LLO fusions at less than 10⁴ molecules. This shift in the TN₅₀ value is consistent with the observed shifts in cytotoxicity when compared to the TN₅₀ for our immunotoxins in the absence of potentiator (˜5×10⁶).

Our experiments showed the potentiation of our gelonin immunotoxins by Fn3-cytolysin fusions also exposed their synergy against antigen negative cells. This discovery further motivated us towards the use of delayed dosing in vivo. To test the possibility of such an approach, we used a delayed exposure assay in which cells were treated with immunotoxin for a fixed period of time followed by exposure to a potentiating agent after increasing delay times. We found that at low concentrations, a 48 hour delay between exposures was sufficient to abrogate potentiated cytotoxicity, but that at higher concentrations, even on cells expressing low levels of antigen, potentiation was still possible and even potent after the same delay. These experiments suggest that, given a sufficient in vivo dose, administration might be delayed by equally long times, which should be sufficient for the first agent to be cleared from the plasma, thereby reducing simultaneous exposure of antigen-negative cells subject to the highest concentrations of either agent.

In vivo studies showed the potential of our combination system as an effective anti-cancer therapy. We identified tolerated doses of each protein individually and tolerated dosing schemes for their combination. The two therapeutics were shown to localize to the tumor and to induce apoptosis, two requirements for in vivo efficacy. The selected dosing regimen was able to control xenograft tumor growth with just two rounds of treatment. However, this same regimen also led to observable toxicity. Because this is the first time this type of synergistic therapeutic interaction has been attempted, there remains a substantial amount of work that needs to be done to optimize dosing. Under a different set of conditions it should be possible to eliminate this non-specific toxicity while maintaining anti-tumor activity.

Applications of this technological approach are as broad as they may be impactful. We believe that many other targeted therapeutic macromolecules with cytoplasmic activity, such as siRNA, will be enhanced by concomitant treatment with Fn3-cytolysin fusions. At the same time, one of ordinary skill in the art will recognize that that these tools will require optimization with available methods at different levels to negotiate residual non-specific toxicities or immunogenicity and to perfect therapeutic efficacy. Specifically, the particular forms of the binding and cytolysin components of the fusion protein may need to be engineered to adjust levels of affinity or activity. In each formulation, the dosing of the potentiating fusion protein and the therapeutic agent will most likely have to be determined on an empirical basis with respect to scale, timing, and frequency. Nevertheless, our results here indicate that this therapeutic approach is viable as a solution to the challenge of targeted intracellular drug delivery.

### Example 3

### A Proteinaceous Multi-Agent System for Potent Targeted Delivery of Small Interfering RNA Using a Non Polycationic RNA Carrier

In the work described below, we present the use of a multi-agent protein based siRNA delivery system for targeted siRNA delivery (see FIG. 6). The first agent, termed E6N2, employs the double stranded RNA binding domain of human protein kinase R (Bevilacqua and Cech, Biochemistry 35:9983-9994, 1996) as an alternative siRNA carrier with low charge density, and an EGFR-binding variant of the 10th type 3 fibronectin (Fn3) for targeting (Hackel et al., J. Mol. Biol. 401:84-96, 2010). Although dsRBD has also been used previously for siRNA delivery, these prior works relied on polycationic cell penetrating peptides for cytoplasmic entry M an untargeted manner (Eguchi et al., Nature Biotechnol. 27:567-571, 2009; Kim et al., J Gene Med. 11:804-812, 2009).

E6N2 is able to deliver siRNA to the endosomes of EGFR-expressing cells. In order to enhance the endosomal escape of siRNA, a second agent is used, consisting of an alternate EGFR-binding Fn3 clone fused to the cholesterol dependent cytolysin, perfringolysin O (PFO) (see FIG. 6; a potentiating moiety). PFO is delivered in a targeted fashion and disrupts endosomal compartments to allow the escape of internalized siRNA to access the cytoplasm. Successful gene silencing is achieved with the two-moiety approach, but the addition of a third moiety that induces EGFR clustering (see FIG. 6) can significantly widen the therapeutic window, through the simultaneous enhancement in gene silencing potency and protection from cytotoxicity of EGFR-binding Fn3-PFO fusion proteins.

Useful Reagents and Methods:

Protein Expression and Purification.

The gene containing the dsRBD moiety from human protein kinase R was a kind gift from Dr. James Cole (University of Connecticut). Genetic fusions containing E6-mouse IgG2a Fc-dsRBD with an N-terminal His tag were constructed and inserted into the gWiz vector (Genlantis, San Diego, Calif.) using the method described by Geiser et al. (Biotechniques 1:88-92, 2001). The C121V and C135V mutations, which were shown not to be important for RNA binding (Spanggord and Beal, Nucl. Acids Res. 28:1899-1905, 2000), were incorporated into dsRBD using the Quikchange mutagenesis kit according to the manufacturer's instructions (Agilent, Santa Clara, Calif.). E6N2 was expressed in transiently transfected HEK293F cells (Invitrogen, Carlsbad, Calif.) for 8 days. E6N2 was purified from the supernatant using a Talon column according to the manufacturer's instructions (Clontech, Mountain View, Calif.).

Fn3-PFO genetic fusions with a C-terminal His tag and a C215A mutation were constructed using a modified Quikchange reaction as described by Geiser et al. (supra) and inserted into the pmal-c2x vector with a TEV cleavage site immediately downstream of the Factor Xa site. In total, 4 fusions with EGFR-binding Fn3's (E6-PFO, C-PFO, D-PFO, E-PFO) and one fusion with a CEA-binding Fn3 (C7-PFO) were constructed (Hackel, supra; Pirie, supra). Fn3-PFO fusion proteins were transformed into Rosetta 2 (DE3) E. coli (Novagen, San Diego, Calif.). Cells were grown to OD₆₀₀=0.5-1.0 and induced with 0.5 mM IPTG for 6 hours at 30° C. Resuspended cell pellets were sonicated and the lysates were subjected to purification on an amylose column according to the manufacturer's instructions (New England Biolabs, Ipswich, Mass.). After overnight digestion with TEV protease, Fn3-PFO proteins were purified by ion exchange chromatography.

The multispecific construct HNB-LCD was prepared as described previously.

Tissue Culture.

The human epidermoid carcinoma cell line, A431, was cultured in a humidified atmosphere in 5% CO₂ in DMEM supplemented with 10% heat-inactivated fetal bovine serum and 1% Pen/Strep. A431 cells stably expressing d2EGFP under the CMV promoter were generated by transfection of pd2EGFP-N1 (Clontech, Mountain View, Calif.) using the Amaxa Nucleofector 2b (Lonza, Germany) according to the manufacturer's instructions. 48 hours after transfection, 0.75 mg/mL G418 (Invitrogen, Carlsbad, Calif.) was added to the culture medium. G418-resistant cells were propagated and the GFP-expressing fraction was sorted twice by FACS using a Mo-Flo sorter (Cytomation, Carpinteria, Calif.). The resulting cells, termed A431-d2EGFP, were >99% GFP-positive and were cultured using a maintenance G418 concentration of 0.1 mg/mL.

Agarose Gel Shift Assay.

50 pmol siRNA was mixed with varying amounts of E6N2 for 30 minutes at room temperature. The resulting complexes were run on a 2% agarose gel and visualized using SYBR-Gold (Invitrogen, Carlsbad, Calif.).

Measurement of dsRBD and siRNA Binding Affinity.

In order to quantify the siRNA binding affinity of the dsRBD portion of E6N2, E6N2 was loaded onto Protein A Dynal Beads (Invitrogen, Carlsbad, Calif.). The Protein A beads were washed in PBS+0.1% BSA (PBSA) and resuspended in DMEM+10% FBS adjusted to the specified pH with Alexa 488 labeled AllStars Negative Control siRNA (Qiagen, Valencia, Calif.) at the specified concentration at 37° C. for 1 hour. The beads were washed twice in ice cold PBSA and analyzed by flow cytometry on an Accuri C6 flow cytometer (Accuri, Ann Arbor, Mich.). The K_{D,app} of binding was numerically calculated from the data as described previously (Liu et al., J. Immunother. 32:887-894, 2009).

siRNA Cell Uptake Assay.

A431 cells were plated in 96 well flat bottom plates and serum starved overnight. Alexa 488 labeled Negative AllStars siRNA was complexed with E6N2 at a 1:1 ratio for 30 minutes at room temperature. Complexes or free siRNA in the absence of E6N2 were added to the cells at a 100 nM final concentration, in complete media (10% FBS), in the presence or absence of varying amounts of HNB-LCD. At each time point, the cells were washed twice with PBS and trypsinized for 20 minutes. Cells were washed twice with complete media and resuspended in PBS+2% FBS for analysis by flow cytometry.

In order to correlate the fluorescence signal with the number of siRNA molecules, a calibration curve was determined using the Quantum Simply Cellular anti-Mouse beads according to the manufacturer's instructions (Bangs Laboratories, Fishers, Ind.), using E6N2 labeled with Alexa 488 at a 6 dye:1 protein ratio.

Fluorescence Microscopy.

Cells were plated on MatTek chambers with a 0.13 mm glass coverslip bottom (Ashland, Mass.), and serum starved overnight. Alexa 488 labeled siRNA was complexed with E6N2 at a 1:1 ratio for 30 minutes at room temperature and added to the cells at a 100 nM final concentration in complete media. After 6 hours, the cells were washed with complete media, and then stained with LysoTracker Red (Invitrogen, Carlsbad, Calif.) and DAPI (Roche Applied Sciences, Indianapolis, Ind.). Cells were imaged using a Delta Vision fluorescence microscope (Applied Precision, Issaquah, Wash.).

GFP Knockdown Assay.

A431-d2EGFP cells were plated in 96 well flat bottom plates and serum starved overnight. E6N2 was mixed with either Negative Control AllStars siRNA or GFP Duplex I siRNA (Thermo Scientific, Lafayette, Colo.) at a 1:1 ratio for 30 minutes at room temperature. Complexes or free siRNA in the absence of E6N2 were added to the cells in complete media with varying amounts of Fn3-PFO, in the presence or absence of 7.5 nM HNB-LCD. After 6 hours, cells were washed and incubated for 24 hours in complete media. The cells were trypsinized, washed twice with PBS+2% FBS and analyzed by flow cytometry.

Cytotoxicity of PFO Fusion Proteins.

Cytotoxicity measurements were performed using the Wst-1 reagent with a 1 hour incubation according to the manufacturer's instructions (Roche Applied Science, Indianapolis, Ind.). They were performed either prior to trypsinization for flow cytometry analysis of GFP expression in GFP knockdown assays or in separate cytotoxicity assays. When performed prior to GFP analysis, the cells were washed twice with PBS after Wst-1 exposure. The presence of d2EGFP did not affect Wst-1 reagent performance. The Wst-1 reagent also did not affect GFP expression measurements in knockdown assays.

Hemolysis Assay.

Fresh mouse red blood cells (Fitzgerald Industries, Acton, Mass.) were washed 3 times in PBSA. 50 μL of a 10% suspension of red blood cells were used per sample. The cells were then incubated for 1 hour at 37° C. with varying amounts of PFO, or 10% Triton-X 100 as a positive control for lysis. The cells were centrifuged for 4 minutes at 14000g and the supernatants were measured for hemoglobin release by absorbance at 541 nm.

Construction of Fc-dsRBD Fusion Proteins Targeted to Alternate Antigens.

N2-IL2 contains the same topology as E6N2, with the exception that instead of the Fn3 clone E6 for EGFR targeting, a murine form of the IL-2 antagonist described previously is used for CD25-specific binding. sm3e-dsRBD contains a genetic fusion of dsRBD to the C terminus of the heavy chain of the CEA binding IgG1, sm3e. Both constructs were cloned into the gWiz vector and expressed in transient transfections of HEK293F cells for 8 days, and were purified from supernatants using Protein A agarose affinity chromatography according to the manufacturer's instructions (Thermo Fisher Scientific, Rockford, Ill.).

Description and Construction of Alternate Multispecific Constructs.

In total, three multispecific constructs developed by Spangler et al. (manuscript submitted) were evaluated for enhancing siRNA uptake. In addition to HNB-LCD, which is described in the main text, HND-LCA and HNB-HCA-LCD were also evaluated. HND-LCA is a tri-specific with Fn3 clone D on the N terminus of the heavy chain and Fn3 clone A on the C terminus of the light chain of cetuximab. HNB-HCA-LCD is a tetra-specific with an additional Fn3 clone A on the C terminus of the heavy chain of HNB-LCD. The preparation of these multispecific constructs have been described previously (Spangler et al., manuscript submitted).

siRNA Potency Calibration Curve.

In order to determine the number of siRNA molecules required for GFP knockdown in A431-d2EGFP cells, Amaxa electroporation was used as a positive control for cytoplasmic delivery. A431-d2EGFP cells were electroporated according to the manufacturer's instructions with varying amounts of either GFP Duplex I siRNA or Negative Control AllStars siRNA. The cells were plated and analyzed for GFP expression by flow cytometry 24 hours post-transfection. To measure the amount of siRNA delivered at each concentration of gfp siRNA, A431 cells were electroporated with identical amounts of Alexa 488 labeled control siRNA. The fluorescence signal was measured by flow cytometry within 1 hour of electroporation. The number of siRNA molecules delivered was determined using a calibration curve generated by the Quantum Simply Cellular beads, as described in the main text.

Measurement of EGFR Binding Affinity of Fn3 Fusion Proteins.

In order to quantify the EGFR binding affinity of E6N2, A431 cells were trypsinized and mixed with varying concentrations of E6N2 at 4° C. for 6 hours. The cells were washed twice with PBSA, and stained with Alexa 488 labeled Goat anti Mouse IgG (Invitrogen, Carlsbad, Calif.) for 20 minutes at 4° C. The cells were washed twice with PBSA and analyzed by flow cytometry with the K_{D} of binding determined as described previously (Spanggord and Beal, Nucl. Acids Res. 28:1899-1905, 2000).

In order to quantify the EGFR binding affinity of E6-PFO, C-PFO, D-PFO and E-PFO, A431 cells could not be used due to PFO binding to cholesterol-containing cell membranes, even at 4° C. Therefore, yeast displaying EGFR ectodomain 404SG were employed (19). The yeast cells were incubated with varying amounts of Fn3-PFO for 6 hours at 4° C., washed twice with PBSA, then stained with rabbit anti His antibody (Abeam, Cambridge, Mass.) labeled with the Alexa 647 Microscale Labeling Kit (Invitrogen, Carlsbad, Calif.). The cells were washed twice with PBSA and analyzed by flow cytometry with the K_{D} of binding determined as described previously (Liu et al., J. Immunother. 32:887-894, 2009).

Results:

Preparation of E6N2.

E6N2 is a homodimeric protein that contains 3 components: E6, an engeineered Fn3 variant that binds EGFR (Hackel et al., supra) for EGFR-specific targeting and internalization; the IgG2a Fc fragment; and the double stranded RNA binding domain (dsRBD) of human protein kinase R for siRNA complexation (Rosado et al., Cell Microbiol. 10:1765-1774, 2008). E6N2 was expressed in transient transfections of HEK293F cells, with a single affinity chromatography purification step. Purification by Protein A or cobalt based resins typically yielded 1-3 mg protein per liter of cell culture, and the resulting protein was monomeric by SDS-PAGE and by size exclusion chromatography analysis. Compared to the refolding or chemical conjugation steps of polycationic peptide fusion constructs (Kumar et al., supra; Winkler et al., supra), dsRBD fusions are well behaved and are relatively straightforward to purify. The EGFR binding Fn3 moiety also retains high affinity binding to EGFR in the E6N2 construct, with apparent dissociation constant, K_{D,app}˜2.1 nM.

Analysis of siRNA/dsRBD Interactions.

In order to visualize the complexation between E6N2 and siRNA, an agarose gel shift assay was performed. A shift in the siRNA band was visible in the presence of E6N2, with partial complexation at a dsRBD:siRNA ratio of 1, based on the partial disappearance of the free siRNA band. Complete complexation is observed at dsRBD:siRNA ratios of 2 and above, indicating potent complexation between the siRNA and dsRBD.

For a more quantitative measurement of the dsRBD/siRNA binding affinity, titrations of fluorescently labeled siRNA were performed on Protein A magnetic beads pre-loaded with E6N2. The titrations were performed in complete media at 37° C. to simulate physiological conditions. Previous reports describe a weak siRNA binding affinity of dsRBD, with K_{D} 200 nM (Bevilacqua and Cech, Biochemistry 35:9983-9994, 1996). In E6N2, dsRBD is bivalent, which enhances the avidity of siRNA binding. The K_{D,app} of siRNA binding was measured to be 3.5±0.2 nM, which represents a significant avidity enhancement from bivalent dsRBD.

pH Sensitivity of Binding of siRNA and dsRBD.

In order for the siRNA cargo to be loaded into the RISC/Ago2 complex in the cytoplasm for gene silencing, it must be able to dissociate from dsRBD. Therefore, the K_{D,app}'s of binding were measured at pH 7.4, 6.5, and 5.5 to determine if siRNA could dissociate at endosomal or lysosomal pH once it is internalized. The K_{D,app} of siRNA/E6N2 binding is pH dependent, with a difference of over 2 orders of magnitude between pH 7.4 and pH 5.5. This indicates that siRNA will be able to dissociate from dsRBD within the acidic conditions of the endosome or lysosome. Secondary staining using (Fab′)₂ fragments against mouse Fc confirmed that equivalent amounts of E6N2 remained bound to Protein A beads after exposure to each pH condition tested.

siRNA Uptake by E6N2.

The amount of fluorescently labeled siRNA taken up by E6N2/siRNA complexes was measured in A431 cells using flow cytometry. In order to differentiate between surface and internalized fluorescence signal, the cells were trypsinized to degrade surface-bound E6N2 and eliminate surface fluorescence. Using a calibration of fluorescence signal to number of siRNA molecules, it was determined that approximately 10⁶ molecules of siRNA is internalized into A431 cells after 6 hours of treatment with E6N2/siRNA complexes. There is negligible internalization of siRNA by fluid-phase pinocytosis in the absence of E6N2. There is also negligible internalization of siRNA by Fc-dsRBD fusions targeted to irrelevant antigens, CD25 or carcinoembryonic antigen (CEA).

When Amaxa electroporation is used as a positive control for delivery directly to the cytoplasm, fewer than 10⁴ molecules of siRNA are required for observable knockdown of GFP protein expression in A431 cells stably transfected with d2EGFP, a destabilized form of GFP with a 2 hour half-life. However, no GFP knockdown is observed in these cells with over 10⁶ molecules of gfp siRNA delivered by E6N2. Fluorescence microscopy reveals that almost all of the detectable internalized siRNA are trapped within endosomal and lysosomal compartments. This implies that endosomal escape is the critical barrier for effective RNAi by siRNA delivered by dsRBD fusion proteins.

PFO Fusion Proteins for Endosomal Escape.

In order to enhance the endosomal escape of endocytosed siRNA delivered by E6N2, fusion proteins were constructed containing the cholesterol dependent cytolysin, PFO, and various EGFR-binding Fn3 clones. In total, four PFO fusion constructs with unique EGFR-binding Fn3 clones were evaluated. For knockdown assays, we used the A431 cell line stably transfected with d2EGFP under the CMV promoter, A431-d2EGFP. When added to A431-d2EGFP cells along with 100 nM E6N2/siRNA complexes, all EGFR-binding Fn3-PFO fusion proteins mediated GFP knockdown in a dose dependent manner, most likely due to enhancement of endosomal escape of endocytosed siRNA. Of these, the fusion with clone “D,” D-PFO, was found to be most promising and was used for further characterization.

D-PFO exhibits high EGFR binding affinity, with K_{D}˜6.6 nM, and PFO lytic activity, as shown in hemolysis assays. Reductions in GFP expression are not due to a global downregulation of all cell proteins, or an artifact of PFO cytotoxicity, because the GFP expression levels are unchanged when negative control siRNA is delivered. GFP knockdown is also dependent on delivery by E6N2, as free gfp siRNA cannot induce GFP knockdown in the presence of D-PFO. The PFO fusion protein with C7, an Fn3 that binds an irrelevant antigen, CEA (Pirie et al., J. Biol. Chem. 286:4165-4172, 2010), is not capable of mediating GFP knockdown. Altogether, these data reveal that gene silencing is dependent on both the delivery of siRNA by E6N2, and EGFR-binding of Fn3-PFO fusion proteins, and is not the result of an alternate mechanism of cytoplasmic delivery, such as siRNA diffusion through pores in the cell membrane formed by PFO, or non-sepcific uptake of PFO fusion proteins into endosomes.

Enhancement of siRNA Uptake by Multispecific Constructs.

Successful gene silencing was achieved through EGFR-specific siRNA internalization mediated by E6N2, and endosomal escape mediated by D-PFO. However, the therapeutic window of this method is relatively narrow. In order to expand this therapeutic window, a third agent that induces EGFR clustering was used. Previously, Spangler et al. showed that antibody-Fn3 fusion proteins that bind to multiple distinct epitopes of EGFR can induce EGFR clustering and downregulation. This results in an increased internalization rate due to the simultaneous internalization of clustered EGFR, which in turn can enhance gene silencing potency due to a concentrating effect of EGFR molecules per endosome. Out of three candidate multispecific constructs, HNB-LCD was the most effective in enhancing E6N2-mediated siRNA uptake, with greater than two fold enhancement of siRNA uptake.

Enhancement of GFP Knockdown by Multispecific Constructs.

Next, HNB-LCD was tested to see if the enhancement in siRNA uptake could result in enhanced knockdown of GFP expression. The enhancement of GFP knockdown depends on the Fn3 clone used, with D-PFO showing the greatest amount of enhancement, approximately 4 fold. GFP expression is not altered when delivering control siRNA by E6N2 in the presence of HNB-LCD. In the presence of 7.5 nM and 100 nM E6N2/siGFP complexes, D-PFO has an EC₅₀<15 pM for GFP knockdown.

Cytotoxicity Profiles of Fn3-PFO in the Presence of HNB-LCD.

It was originally hypothesized that the addition of a multi-epitopic EGFR binder would only enhance GFP knockdown through clustering, without any effect on PFO-related cytotoxicity. However, when the cytotoxicity profiles were measured for the various Fn3-PFO fusion proteins, it was found that the presence of HNB-LCD had a protective effect on A431 cells. This effect was consistent across all EGFR-binding Fn3-PFO constructs, and provided an effective 3-4 fold reduction in Fn3-PFO cytotoxicity. When the cytotoxicity of CEA-binding C7-PFO was measured in A431 cells, there was no difference in the presence or absence of HNB-LCD. This indicates that the reduction in Fn3-PFO cytotoxicity by HNB-LCD requires EGFR binding by the Fn3-PFO construct.

Potency of E6N2 for Gene Silencing.

With 100 pM D-PFO, a sub-cytotoxic concentration, and 7.5 nM HNB-LCD, the potency of E6N2-mediated gene silencing was measured at a constant 1:1 ratio of E6N2/siGFP. GFP knockdown was observed at all concentrations of E6N2 greater than the measured K_{d,app} of E6N2 binding to EGFR. Greater than 50% GFP knockdown was observed at E6N2 concentrations of 16 nM and greater.

Discussion:

The dsRBD moiety is expressed and purified in a straightforward manner, without any chemical conjugation required. In our experience, it was not prone to aggregation, presumably due to its relative low charge density, unlike the highly-charged polycationic peptides used previously for siRNA complexation ((Kumar et al., Cell 134:577-586, 2008; Song et al., Nat. Biotechnol. 23:709-717, 2005; Peer et al., Proc. Natl. Acad. Sci. USA 104:4095-4100, 2007; and Winkler et al., Mol. Cancer. Ther. 8:2674-2683, 2009). The dsRBD moiety also binds reversibly and specifically to double stranded RNA, and provides protection against siRNA degradation by RNases (Kim et al., J. Gene Med. 11:804-812, 2009). Significantly, the dsRBD moiety interacts with the RNA backbone in a double-stranded RNA dependent and sequence-independent manner, thus allowing siRNA directed against any target to be loaded (Bevilacqua and Cech, Biochemistry 35:9983-9994, 1996). In combination with PFO-fusions, dsRBD fusion proteins deliver enough siRNA to the cytoplasm for potent gene silencing. As low as 16 nM siRNA can induce >50% gene silencing, whereas typically, 1 μM or more is used with polyarginine as an siRNA carrier (Kumar et al., Cell 134:577-586, 2008) and 100 nM-5 μM is used with protamine as an siRNA carrier (Song et al., Nat. Biotechnol. 23:709-717, 2005; Peer et al., Proc. Natl. Acad. Sci. USA 104:4095-4100, 2007; and Winkler et al., Mol. Cancer. Ther. 8:2674-2683, 2009). These properties make dsRBD fusion proteins an attractive option to other peptide-based methods for complexing siRNA, however other peptide-based RNA-binding proteins can also be incorporated in the therapeutic moieties of the present invention.

PFO fusion proteins are effective at enhancing endosomal escape of siRNA delivered by dsRBD fusions in an EGFR-targeted manner. Their potencies are quite remarkable, considering that GFP knockdown is achievable at Fn3-PFO concentrations less than the K_{D} of EGFR binding, even though Fn3-PFO binding to EGFR is required. On the other hand, despite its potency, E6N2 must be used at concentrations greater than the K_{D,app} of EGFR binding for effective siRNA delivery. Therefore, although there is added complexity arising from the use of two agents, the difference in the effective concentrations of E6N2 and Fn3-PFO and the cytotoxic limits of Fn3-PFO may support the use of the two agents separately, as opposed to a single agent containing both functions in the form of a fusion protein.

The use of multispecific antibody-Fn3 fusion constructs was motivated by the prospect of enhancing gene silencing potency by inducing EGFR clustering and increasing the number of EGFR internalized per endosome. Indeed, HNB-LCD is capable of enhancing gene silencing, though the degree of enhancement is sensitive to the particular clone used in Fn3-PFO fusion constructs. The use of multispecific constructs was not hypothesized to have any effect on PFO-mediated cytotoxicity. The observation that HNB-LCD can reduce the cytotoxicity of EGFR-binding Fn3-PFO constructs was indeed surprising, especially since EGFR downregulation induced by clustering has been shown to reduce viability (Spangler). The exact mechanism for this protective effect has yet to be elucidated. Initially, we hypothesized that the downregulation induced by HNB-LCD resulted from decreased exposure to epidermal growth factor from FBS in the media, which has been shown to inhibit growth in A431 cells (Barnes, J. Cell Biol. 93:1-4, 1982). If this were the case, the protective effect by HNB-LCD would be independent of the Fn3-PFO construct used. However, when using the CEA-binding C7-PFO, no difference in cytotoxicity is observed in the presence or absence of HNB-LCD. This indicates that HNB-LCD can only reduce the cytotoxicity of EGFR-binding Fn3-PFO constructs.

When inducing EGFR downregulation with HNB-LCD, EGFR-bound Fn3-PFO will likely be co-internalized. At the Fn3-PFO concentrations used, the number of EGFR molecules and Fn3-PFO molecules is the same order of magnitude, and thus, it is possible that increasing the EGFR internalization rate by receptor clustering would more rapidly deplete extracellular Fn3-PFO. It is believed that cytotoxicity of Fn3-PFO is based on cell membrane disruption, as opposed to endosomal disruption. This is based on the similarities in A431 cytotoxicity of Fn3-PFO, regardless of EGFR binding, as well as hemolysis of EGFR-negative red blood cells. Therefore, it is believed that the reduction in cytotoxicity from EGFR clustering and downregulation induced by HNB-LCD is due to decreased extracellular Fn3-PFO available for plasma membrane disruption.

The addition of HNB-LCD to E6N2/siRNA and D-PFO significantly expands the therapeutic window, with approximately 2 orders of magnitude difference in the half maximal lethal dose of D-PFO and the half maximal effective dose of D-PFO for GFP knockdown. This arises from both the enhancement of GFP knockdown and the decrease in D-PFO toxicity by HNB-LCD. As a therapeutic modality, a third agent would significantly increase the complexity of treatment, and that is why the invention encompasses, but is not limited to, therapeutic moieities and potentiating moieties that incorporate multispecific binding agents.

1. A potentiating moiety comprising (a) a binding agent that specifically binds a cell surface molecule, (b) a lytic agent that destabilizes the membrane of an intracellular compartment and (c) optionally, a linker between the binding agent and the lytic agent.

2. The potentiating moiety of claim 1, wherein the binding agent is a tenth Type III fibronectin domain engineered to specifically bind the cell surface molecule and the lytic agent is listeriolysin O, a homolog thereof, or a biologically active variant thereof.

3. The potentiating moiety of claim 1, wherein the cell surface molecule is a tyrosine kinase receptor or a tumor antigen.

4. The potentiating moiety of claim 1, wherein the binding agent and the lytic agent are joined as a fusion protein or a chemical conjugate.

5. The potentiating moiety of claim 1, further comprising a therapeutic agent.

6. The potentiating moiety of claim 1, wherein the moiety comprises a plurality of binding agents that bind more than one distinct epitope on a cell surface molecule, such that the moiety as a whole is multi-specific and constitutes a cluster-inducing therapeutic moiety further including a lytic agent.

7. A cluster-inducing therapeutic moiety comprising a therapeutic agent and a plurality of binding agents that bind more than one distinct epitope on a cell surface molecule.

8. A cluster-inducing potentiating moiety comprising a lytic agent and a plurality of binding agents that bind more than one distinct epitope on a cell surface molecule.

9. The potentiating moiety of claim 1, wherein the moiety is configured as a fusion protein.

10. A therapeutic moiety designed to deliver an RNA-based therapeutic comprising (a) a binding agent that specifically binds a cell surface molecule, and (b) a non-polycationic carrier that reversibly binds double- or single-stranded RNA.

11. The therapeutic moiety of claim 10, wherein the non-polycationic carrier is selected from the group consisting of a double-stranded RNA binding domain (dsRBD), a translation initiation factor, an snRNP, and ADAR.

12. The therapeutic moiety of claim 11, wherein the non-polycationic carrier is a dsRBD.

13. The therapeutic moiety of claim 10 further comprising an accessory sequence selected from the group consisting of a linker or an Fc region.

14. The therapeutic moiety of claim 13 comprising a fusion protein comprising an Fc region, wherein the binding agent and non-polycationic carrier are fused to distinct sites of the Fc region.

15. The therapeutic moiety of claim 14, wherein the binding agent and non-polycationic carrier are each fused to the Fc region via a flexible linker.

16. The therapeutic moiety of claim 10, further comprising an RNA-based therapeutic.

17. The therapeutic moiety of claim 10, wherein the binding agent is selected from the group consisting of an antibody or antibody fragment, a growth factor and a fibronectin domain.

18. The therapeutic moiety of claim 10, wherein the binding agent is a type III fibronectin (Fn3) domain comprising BC, DE, and FG loops.

19. The therapeutic moiety of claim 18, wherein the cell surface molecule is selected from the group consisting of EGFR, CEA and CD25.

20. The therapeutic moiety of claim 19, wherein the Fn3 domain comprises BC, DE, and FG loops of E6N2.

21. The therapeutic moiety of claim 10, further comprising a therapeutic RNA molecule.

22. A nucleic acid comprising a sequence that encodes the fusion protein of claim 9.

23. The nucleic acid of claim 22, wherein the nucleic acid encodes a fusion protein comprising, from the N-terminus to the C-terminus: maltose binding protein, N₁₀ linker, a Factor Xa protease site, the tenth Type III fibronectin domain, a G₄S linker, and listeriolysin O or a biologically active variant thereof.

24. An expression vector comprising the nucleic acid sequence of claim 22.

25. The expression vector of claim 24, wherein the expression vector is a plasmid.

26. A host cell comprising the expression vector of claim 24.

27. The host cell of claim 26, wherein the host cell is a prokaryotic cell.

28. The host cell of claim 27, wherein the prokaryotic cell is E. coli.

29. A kit comprising the potentiating moiety of claim 1 and instructions for use.

30. A pharmaceutical composition comprising the potentiating moiety of claim 1.

31. A method of delivering a therapeutic agent to a cell, the method comprising administering to a patient in need of the therapeutic agent a therapeutically effective amount of: (a) the potentiating moiety of claim 1 and (b1) a therapeutic moiety comprising a binding agent that specifically binds a cell surface molecule, a non-polycationic carrier that reversibly binds double- or single-stranded RNA, and a therapeutic agent, or (b2) a therapeutic moiety that is free of a binding agent.

32. The method of claim 31, wherein the patient has cancer and the binding agent specifically binds a cancer cell-associated antigen and the therapeutic agent is a chemotherapeutic agent.

33-34. (canceled)
